(19) United States
(12) Patent Application Publication
(10) Pub. No.: US 2026/0196343 A1
(43) Pub. Date: Jul. 9, 2026

(54) MANAGING CONCURRENT BREAKS

(71) Applicant: MEDTECH INNOVATIONS LLC, Rockville, MD (US)

(72) Inventor: Marc Neubauer, Rockville, MD (US)

(21) Appl. No.: 19/551,591

(22) Filed: Feb. 26, 2026

Related U.S. Application Data
(63) Continuation-in-part of application No. 18/639,861, filed on Apr. 18, 2024.
Application No. 18/639,861 is a continuation-in-part of application No. 17/745,041, filed on May 16, 2022, now Pat. No. 12,198,529.
Application No. 17/745,041 is a continuation-in-part of application No. 17/506,673, filed on Oct. 21, 2021, now Pat. No. 11,380,186.
(63) Continuation of application No. PCT/US2025/042598, filed on Aug. 19, 2025.
(60) Provisional application No. 63/685,202, filed on Aug. 20, 2024.
(60) Provisional application No. 63/841,446, filed on Jul. 10, 2025.

(51) Int. Cl. G16H 40/20; G06Q 10/0631

(52) U.S. Cl. CPC G16H 40/20; G06Q 10/06311

(57) ABSTRACT

Disclosed are methods and apparatuses for managing a concurrent break. An example method includes selecting one or more first clinicians to take a concurrent break. The example method can identify a total pool of patients that need to be covered and select one or more second clinicians that have a set of break coverage attributes. The method can assign one or more of the total pool of patients to each of the selected second clinicians while complying with their respective break coverage attributes. Once all patients are covered, the selected first clinicians can be notified of the patients that they are assigned to cover during the concurrent break.

### CROSS-REFERENCE TO RELATED APPLICATIONS

This application is a continuation-in-part of U.S. Non-Provisional application Ser. No. 18/639,861 filed on Apr. 18, 2024, which is a continuation-in-part of U.S. Non-Provisional application Ser. No. 17/745,041 filed on May 16, 2022, now U.S. Pat. No. 12,198,529, which is a continuation-in-part of U.S. Non-Provisional application Ser. No. 17/506,673 filed on Oct. 21, 2021, now U.S. Pat. No. 11,380,186. This application is also a continuation of International Patent Application No. PCT/US2025/042598 filed on Aug. 19, 2025, entitled “MANAGING CONCURRENT BREAKS”, which claims priority to U.S. Provisional Patent Application No. 63/841,446 filed on Jun. 10, 2025 and U.S. Provisional Patent Application No. 63/685,202 filed on Aug. 20, 2024. The contents of all the applications referenced in this section are hereby incorporated in their entirety and for all purposes.

### TECHNICAL FIELD

The present invention generally relates to assignment and scheduling of tasks and breaks to staff members and, in particular, enabling multiple clinicians to take a concurrent break.

### BACKGROUND

Conventional systems and methods of managing staff member schedules that include breaks are largely manual and often limited to enabling only a single staff member to take a break. Certain facilities hire a Break Nurse whose role is solely to cover the patients of a single nurse while that nurse takes a break. This simple approach does not enable multiple nurses to take breaks that overlap.

### SUMMARY

What is needed is an automatic system that collects information about the patients assigned to clinicians and determine whether there is sufficient collective bandwidth to distribute patients from a first plurality of clinicians across a second plurality of clinicians, thereby enabling the first plurality of clinicians to go on a concurrent break. The systems and methods described herein fulfill this need.

### BRIEF DESCRIPTION OF THE DRAWINGS

Aspects of the disclosure are herein described, by way of example only, with reference to the accompanying drawings. With specific reference now to the drawings in detail, it is stressed that the particulars shown are by way of example and are for purposes of illustrative discussion of aspects of the disclosure. The description and the drawings, considered alone and together, make apparent to those skilled in the art how aspects of the disclosure may be practiced.

FIG. 1 depicts an exemplary workflow for managing a break for an individual, according to certain aspects of the present disclosure.

FIG. 2 depicts an exemplary workflow for managing options for giving a staff member a break, according to certain aspects of the present disclosure.

FIG. 3 depicts an exemplary workflow for enabling a staff member to control their own break schedule, according to certain aspects of the present disclosure.

FIG. 4 depicts an exemplary workflow for managing a break schedule for staff members in a care unit, according to certain aspects of the present disclosure.

FIG. 5 depicts an exemplary workflow for assigning a task in response to an initiating event, according to certain aspects of the present disclosure.

FIG. 6 depicts an exemplary workflow for reducing fatigue in a staff member, according to certain aspects of the present disclosure.

FIG. 7 depicts an exemplary workflow for re-assigning a task from one staff member to another staff member, according to certain aspects of the present disclosure.

FIG. 8 depicts an exemplary workflow for handing off tasks from one staff member to another staff member, according to certain aspects of the present disclosure.

FIG. 9 depicts an exemplary system for managing tasks and breaks, according to certain aspects of the present disclosure.

FIG. 10 depicts a flowchart of an exemplary method of managing breaks, according to certain aspects of the present disclosure.

FIG. 11 depicts a flowchart of an exemplary method of optimizing selected characteristics of the schedule, according to certain aspects of the present disclosure.

FIGS. 12-14 depict exemplary user interface screens, according to certain aspects of the present disclosure.

FIGS. 15-16 are tables containing exemplary weightings used to determine who goes on break and who covers for each break, according to certain aspects of the present disclosure.

FIG. 17 depicts an exemplary user interface screen displaying unit and key individual performance, according to certain aspects of the present disclosure.

FIG. 18 depicts exemplary recommendations, according to certain aspects of the present disclosure.

### DETAILED DESCRIPTION

This description is intended to illustrate some particular embodiments of the disclosure and not to exhaustively specify all permutations, combinations and variations thereof. Features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure and do not depart from the instant disclosure. In some instances, well-known structures, interfaces, and processes have not been shown in detail in order not to unnecessarily obscure the invention. It is intended that no part of this specification be construed to effect a disavowal of any part of the full scope of the invention.

Unless otherwise defined herein, all technical and scientific terms used herein have the same meaning as commonly understood by one of ordinary skill in the art to which this disclosure belongs. The terminology used in the description of the disclosure herein is for the purpose of describing particular aspects or embodiments only and is not intended to be limiting of the disclosure. References to techniques employed herein are intended to refer to the techniques as commonly understood in the art, including variations on those techniques or substitutions of equivalent techniques that would be apparent to one of skill in the art.

As used in this disclosure, the phrase “concurrent break” means a time period wherein one or more clinicians are simultaneously taking a break. This includes time periods wherein a plurality of clinicians are taking breaks with common start times and/or common end times as well as time periods wherein the breaks of two or more clinicians partially overlap.

As used in this disclosure, the term “covered” and the like means that a second clinician is temporarily providing care for a patient assigned to a first clinician, e.g., while the first clinician is on a break or assigned a priority task that prevents the first clinician from being able to provide care to the patient. In certain embodiments, coverage of a patient by the second clinician comprises only monitoring the patient and responding to a new condition, e.g., a medical device alarm, that requires action while the first clinician is unavailable. In other embodiments, coverage of a patient by the second clinician comprises completing tasks associated with the patient (e.g. medical administration, vital signs monitoring, etc.) during the break.

Unless the context indicates otherwise, it is specifically intended that the various features of the disclosure described herein can be used in any combination. Moreover, the present disclosure also contemplates that in some embodiments of the disclosure, any feature or combination of features set forth herein can be excluded or omitted.

The methods disclosed herein comprise one or more steps or actions for achieving the described method. The method steps and/or actions may be interchanged with one another without departing from the scope of the present disclosure. In other words, unless a specific order of steps or actions is required for proper operation of the embodiment, the order and/or use of specific steps may be modified without departing from the scope of the present disclosure.

### Task and Break Management

While the disclosure and examples provided herein are directed to a healthcare environment, e.g., a hospital with care units staffed by nurses and other staff members such as physicians, pharmacists, certified nursing assistants, break nurses, respiratory therapists, radiologic technicians, biomedical engineers, the systems and methods are equally applicable to other environments where the assigned workload and work tasks are subject to change and must be apportioned among a group of staff members. These traits are particularly common in environments that are fast-paced, have variable demand or require teamwork to manage workflows efficiently. For example, the disclosure and examples provided herein can be applied to a retail pharmacy where pharmacists work long hours with limited breaks, where tasks are subject to change. Other applicable fields include retail and wholesale shipping and distribution centers, IT and customer call centers, and software development teams working in fast paced, AGILE environments.

Nurses often skip breaks out of a concern that there may be a gap in the care provided to their assigned patients. While another nurse may be assigned to “cover” for the nurse while on break, the cover nurse has their own set of patients and tasks to accomplish and may not be able to perform important tasks or respond to unscheduled events such as a medical device alarm. This is particularly a problem with the medical-surgical units of a hospital, where nurses often have demanding patient-staff ratios, e.g., 8:1 or even 10:1, and do not have dedicated breaks nurses to cover their breaks. This situation is exacerbated if the unit is short-staffed but there may be little, if any, tangible data to document an overload of the staff. As a result of not taking all their allocated breaks, nurses will experience an increased level of fatigue that may lead to a reduction in the quality of care that they provide, a reduction in their wellbeing, or a reduction in their job satisfaction and job retention status.

The systems and methods disclosed address problems of conventional methods by introducing an automatic system that collects information about the assigned tasks, for example the time windows in which each task should be completed and the time that it takes to perform each task, as well as a compilation of the breaks that should be taken on a shift, for example the number and duration of the breaks. Using rules and constraints that are based on one or more of regulations, hospital policies, staff member break preferences, and best practices, the system compares candidate schedules to identify a schedule for a nurse for their shift in which tasks can be reassigned between different staff members and be accomplished without delay of care and every break can be taken at a defined start time for a defined duration. If it is not possible to execute on a scheduled break on first analysis, the system can modify the dynamic attributes disclosed herein, e.g. break dynamic attributes, and/or reassign tasks based on dynamic attributes to provide the staff member with a break. If the modification of dynamic attributes or reassignment of tasks does not address the situation, then the system identifies the conflicts and provides documentation of the task overload or other factors that prevent the nurse from having enough time to complete the assigned tasks while also taking enough break time.

### Dynamic Attributes

As used within this disclosure, the phrase “dynamic attribute” refers to all changeable factors associated with a patient, a staff member, a task, a shift or a break. Dynamic attributes consider the changeable factors for an individual entity, e.g., a shift, a staff member; and between different entities, e.g., between one shift and another, between staff member A and staff member B.

Break dynamic attributes are attributes that can change on a break or between two different breaks. They may influence how breaks are scheduled and how break schedules are executed to ensure staff members receive their breaks. Break dynamic attributes include, but are not limited to, a start time, a duration, a break type, e.g., a meal break or a training session, a break priority, a second staff member to share the break with, a location of the break, an item to bring to break, e.g., training material, number of breaks per clinician per shift, a break preference of a staff member, e.g. staff member A prefers early meal breaks, and what type of tasks to reduce and what type of tasks to still assign during a period defined as a break.

Shift dynamic attributes are defined as attributes that can change on a shift or between one shift and another. Shift dynamic attributes may influence how breaks are scheduled and/or executed on a shift or how tasks are reassigned on shift. Shift dynamic attributes include, but are not limited to, staffing, shift hours, type of shift (e.g., day shift or night shift), ADT information, type and severity of medical conditions treated during shift, patient/staff assignments ratios, acuity of patients on a given shift, stability of patients on a given shift, number of overtime (OT) hours scheduled and/or worked by staff on a unit during a shift, availability of support staff (e.g., certified nursing assistants and/or break nurses), number of breaks to be scheduled, breaks hour to be scheduled, number of breaks missed or interrupted on a prior shift, community events near hospital during shift, and weather and traffic events during shift. In certain embodiments, shift dynamic attributes include outstanding tasks that need to be carried over from the previous shift to the shift being planned.

Task dynamic attributes are defined as attributes that can change with a task or between two different tasks. Task dynamic attributes may influence how tasks are assigned or reassigned to different staff members and how tasks are communicated to staff on task lists or reports, e.g., break transfer summary reports. Task dynamic attributes include but are not limited to, task priority, tasks completed, time of completion, staff member assigned task, staff member who completed task, outstanding tasks, origination and received times of outstanding tasks, priority of outstanding tasks, and notes or instructions associated with tasks, e.g. instruction to double verify certain medical administration tasks.

Patient dynamic attributes are defined as attributes that can change with a patient or between two or more patients. Patient dynamic attributes may influence a task priority and how tasks are assigned, reassigned, or escalated to different staff members. Patient dynamic attributes include, but are not limited to, the examples listed in Table 1.

TABLE 1

| | |
|---|---|
| FACTOR | EXAMPLE |

Patient Monitor Factors (currently being measured or periodically assessed)

| | | | |
|---|---|---|---|
| 1 | | patient health | |
| | a. | a patient health measurement | vital signs, labs, point of care |
| | | | parameter, diagnostic test result |
| | b. | change in the score of a patient | Modified Early Warning Score |
| | | health assessment | (MEWS), Acute Physiology and |
| | | | Chronic Health Evaluation |
| | | | (APACHE) 2 |
| 2 | | an action by the patient | |
| | a. | detection of motion | patient motion detected directly at |
| | | | same time as alarm known to be |
| | | | induced by motion |
| | b. | loss of signal; abnormal signal | patient motion disconnects an EKG |
| | | | lead causing alarm for low pulse rate |

Identification, Treatment, and Diagnostic Factors (from a hospital system)

| | | | |
|---|---|---|---|
| 3 | | a doctor's order | administer a medication (dose, |
| | | | method, frequency, etc.), specific |
| | | | settings of a medical device |
| 4 | | a patient instruction | a Do Not Resuscitate (DNR) order |
| 5 | | patient medical record | |
| | a. | a patient identifier | Age, gender, ethnicity, blood type, |
| | | | height, weight |
| | b. | a diagnosis of an underlying | heart disease, congenital heart defect, |
| | | condition of the patient | diabetes |
| | c. | an inherent characteristic of the | a genetic marker, an inherited |
| | | patient | condition, e.g., patient is a carrier of |
| | | | hemophilia |
| | d. | whether patient has received a | an implanted device |
| | | treatment that affects a condition | |
| | e. | whether a patient has previously | note in electronic medical record |
| | | experienced a symptom for a | (EMR) of fainting, dizziness, nausea |
| | | condition | |
| | f. | changes in a patient's condition | worsens or improves after an |
| | | | intervention |

Medication Factor (from medication library in a hospital system or standalone medication
library)

| | | | |
|---|---|---|---|
| 6 | | an indication for use of | Vasopressors are indicated to raise |
| | | a medication or fluid being | blood pressure (BP) |
| | | administered to the patient; | |
| 7 | | characteristics of medication or | |
| | | fluid | |
| | a. | intended effect | blood thinning medications indicated |
| | | | for patients with atrial fibrillation to |
| | | | reduce chance of stroke |
| | b. | side effect | thyroid medication can cause very |
| | | | fast heart rate and irregular heart |
| | | | rhythm |
| | c. | patient health measurement | vasopressors increase blood pressure, |
| | | affected by a medication | insulin lowers blood sugar |
| | d. | pharmacokinetic or | PK-e.g., biological half-life |
| | | pharmacodynamic (PK/PD) | PD-e.g., drug-drug interactions |
| | | characteristic | |

Medical Device Factors (configuration or operational performance)

| | | |
|---|---|---|
| 8 | a setting of the medical device | delivery rate of an IV fluid; oxygen |
| | | content of delivered air |
| 9 | an operating mode of the medical | running on battery power, ventilator |
| | device | set to allow spontaneous breathing |
| 10 | an operational status of the | infusion pump has stopped infusing a |
| | medical device | fluid; a ventilator has stopped |
| 11 | a measured aspect of operation of | Measured battery voltage (indication |
| | the medical device | of remaining operational time), tidal |
| | | volume of each breath |
| 12 | whether there is a secondary | tidal volume sensing as secondary |
| | mechanism to mitigate harm | indication of respiration if airway |
| | associated with alarm | pressure alarm occurs, back-up |
| | | battery |
| 13 | A feature of a device | Electrocardiogram (ECG) filter mode, |
| | | number of leads used for monitoring |
| 14 | number of previous | high number indicates artifact, |
| | nonactionable or false alarms | reliability issues, or environmental |
| | received before alarm received | issues, e.g., electromagnetic |
| | | interference |
| 15 | estimated time to perform a | replace a non-operational ventilator or |
| | corrective action | pump |
| 16 | recommended replacement | ECG leads should be changed 1x or |
| | interval of a consumable | 2x per day to reduce false alarms |

As used within this disclosure, the phrase “patient health measurement” refers to one or more of the following patient characteristics: a vital sign, a point-of-care (POC) observation or symptom, a diagnostic test, or a laboratory test. These measurements include but are not limited to body temperature, heart rate, blood pressure, respiration rate, tidal volume, blood oxygenation, blood glucose level, an arrhythmia, and end-tidal carbon dioxide (ETCO2).

As used within this disclosure, the phrase “laboratory test” refers to analysis of a biological sample from the patient, e.g., blood. The analysis may be performed at the bedside or at a remote location.

As used within this disclosure, the phrase “patient health assessment” is an algorithm that may be used to categorize a patient's health or cognitive status, to triage a patient, or to monitor a patient to determine if their condition is improving or worsening. A patient health assessment may be a nationally or locally recognized set of criteria or algorithm or one defined and implemented by a hospital or network of hospitals. For example, a patient's acuity status may be evaluated using the Oulu Patient Classification System. The complexity of a patient's condition may be evaluated using the Charlson Comorbidity Index (CCI) or the Patient Clinical Complexity Level (PCCL) algorithms. Other examples of patient health assessments include a patient pain algorithm, a patient disease severity algorithm, a patient awareness algorithm, a patient agitation algorithm, a patient deterioration algorithm, and a mortality score such as the APACHE 2.

Staff dynamic attributes are defined as attributes that can change with a staff member or between two or more staff members. Staff dynamic attributes may influence how tasks are assigned, reassigned, or escalated to different staff members, how breaks are scheduled, or how breaks are executed. Staff dynamic attributes can be broadly classified into groups based on the following attributes of a staff member: capability to respond to an alarm, availability to respond to a task, current workload status, burnout or fatigue status (i.e., both current and long-term), performance (over a defined period), the management or level of support of a staff member, and the facility in which a staff member works. Dynamic attributes associated with a staff member include, but are not limited to, the examples listed in Table 2.

TABLE 2

| | | ATTRIBUTE | EXAMPLE |
|---|---|---|---|
| 1 | | a capability attribute of a staff member | |
| | a | Age | 30 y/o, 60 y/o |
| | b | Education | licensed practical nurse, registered nurse, |
| | | | physician |
| | c | Experience | five years of experience in an intensive care unit |
| | d | training | training on a specific brand of infusion pumps, |
| | | | ventilators, etc. training on soft skills, |
| | | | communication, etc. |
| | e | certifications | clinical certifications, soft skills certifications |
| | f | a score associated with an education | 4.0 GPA, 95% on a training, etc. |
| | | level, training, certification | |
| | g | frequency of training | staff member who just took the refresher training |
| | h | physical capability | impaired movement, not able to lift > X pounds |
| | i | a psychometric status of a staff member | aptitude test, skill test, personality test |
| | j | a physical or technical capability | an alarm that requires a physical capability (e.g. |
| | | required for a specific alarm | responding to a patient fall alarm), an alarm that |
| | | | requires a technical capability (e.g. a ventilator |
| | | | alarm that requires troubleshooting and changing |
| | | | parameters) |
| 2 | | an availability attribute of a staff member | |
| | a | a break status of the staff member | on break, working |
| | b | a distance from the staff member to the | distance too far to respond to alarm |
| | | medical device | |
| | c | do not disturb status | working a code |
| | d | staff member closest to patient alarm | closest by feet, second closest by feet |
| | e | an availability status based on the need | staff member wears personal protective |
| | | for staff member to implement a special | equipment and is not available to respond to |
| | | control | alarm in non-special control section |
| 3 | | a workload status attribute of a staff member | |
| | a | a number of patients assigned to the staff | 5:1 ratio |
| | | member; | |
| | b | a number of patients having a health | number of patients with high acuity, stability, |
| | | assessment (HA) score above a threshold | complexity, deterioration scores |
| | c | an aggregate health assessment (HA) | total scores for patients assigned to staff member |
| | | score of assigned patients above a | |
| | | threshold | |
| | d | largest change in health assessment | health assessment score for a patient significantly |
| | | scores of assigned patients in a given | increases indicating deterioration |
| | | time period | |
| | e | a number of active alarms associated | three active high priority alarms, 2 active H2 |
| | | with assigned patients assigned a sub- | alarms |
| | | priority or priority above a threshold | |
| | f | a number of scheduled tasks in current | five active nurse calls, 4 infusions ordered |
| | | time period | |
| | g | a number of potential device tasks in | 14 active infusions assigned to current patients, 5 |
| | | current time period | active infusions with critical medications, 10 |
| | | | critical devices that can alarm, 25 total devices |
| | | | that can alarm |
| | h | currently caring for a patient who | patient who is at high risk for falling or exiting |
| | | requires special care | bed (e.g. combative or dementia patient), |
| | | | ventilated patient at high risk of extubating, etc. |
| 4 | | a burnout status attribute of a staff member | |
| | a | a reported level of staff burnout or | monthly job satisfaction score,in-shift emotional |
| | | satisfaction | exhaustion scoresurvey stating clinician needs a |
| | | | vacation or new job |
| | b | a grief event associated with the staff | divorce, death of a loved one |
| | | member | |
| | c | a measurement of the staff member | blood pressure, number of steps, electrical activity |
| | | | of the brain indicating amount of sleep or activity, |
| | | | a lab value, a measure for medication compliance |
| | d | a self-reported value or activity | the number of hours slept before shift, medication |
| | | | compliance |
| | e | a work schedule | staff on overtime hours, number of shifts worked |
| | | | without a day off, number of night shifts per |
| | | | week, number of shifts before a vacation |
| | f | a number of hours worked by staff | number of hours worked on current shift, number |
| | | member per time period | of hours worked in last X days, number of hours |
| | | | worked since last vacation |
| | g | a percentage of time where staff's | 90% of a staff member's shift, 80% of staff's |
| | | workload status = high | shifts in last two weeks |
| | h | a number of device tasks performed per | responded to 85 alarms in last 12 hours, |
| | | a given time period | |
| 5 | | a performance attribute of a staff member | |
| | a | a number of “never” events associated | two never events in last 30 days |
| | | with the staff member per time period | |
| | b | a number of preventable adverse events | three preventable adverse events in last 30 days |
| | | associated with the staff member per | |
| | | time period | |
| | c | a number of adverse events associated | five adverse events in last 30 days |
| | | with the staff member per time period | |
| | d | number of patient related complaints | three patient related complaints in last 30 days |
| | e | a number of alarm responses > response | five high priority alarm responses > goal in last |
| | | time goal per a time period per time | 30 days |
| | | period | |
| | f | a performance score/rating given by a | a rating of 3 out of 5 for communication, |
| | | supervisor | teamwork, clinical ability etc. |
| 6 | | an attribute of management of the staff member | |
| | a | a number of backups to the staff member | 2 backups/staff member |
| | b | whether the staff member is assigned as | yes/no |
| | | a back-up to another staff member | |
| | c | an attribute of the backups | a capability, a workload status, etc. |
| | d | an overall ratio of patients to backups | 12 to 1 |
| | e | type of facility staff is working | intensive care unit, medical/surgical floor |
| | f | a maximum patient-to-staff ratio of the | 3:1 for an ICU |
| | | facility | |
| | g | number or hours or shifts a new staff | first shit, 3 shifts on new team |
| | | member is on a team, unit, etc. | |
| | h | a conflict between staff members | Staff Member A does not get along with Staff |
| | | | Member B |
| | i | a conflict between staff member and | a patient shows hostility to Staff Member A |
| | | patient or family member | |
| | j | a technology available in the facility to | cameras used for dementia patients to notify for |
| | | assist a staff member | potential fall risk |

In certain embodiments, the attributes listed in Table 2 are associated with the groups in the table. In other embodiments, attributes classified in one group, e.g., management of staff, are classified into a different group, e.g., burnout status of a staff member. For example, a conflict between two staff members working together on a shift as depicted in the attributes of the management of a staff member group can contribute to a burnout status score and be classified under this group of attributes.

As used within this disclosure, the phrase “adverse event” identifies an undesirable experience associated with the use of a medical product in a patient that could result in harm, e.g., death or injury or prolonged hospitalization, even when the care is not the cause of the occurrence.

As used within this disclosure, the phrase “preventable event” identifies an adverse event that happens, or is made possible to happen, because of either an error of commission or an error of omission made by a care provider. The error is rarely due to negligence. The error may be an inaccurate or incomplete diagnosis of a disease or an improper execution of a treatment procedure.

Errors have also been attributed to issues in communication, complexity of technology used in treatment, and the use of increasingly powerful drugs.

As used within this disclosure, the phrase “never event” identifies an event that should never happen to a patient. Examples include a wrong-site surgery, a transfusion with the wrong blood type, development of a pressure ulcer, a fall, and a hospital-acquired infection.

As used within this disclosure, the phrase “grief event” identifies an event having a lasting emotional impact and can affect the staff member's ability to do their job. This may be a personal event, e.g., a divorce or loss of a family member, or a community event, e.g., loss of a coworker or closing or a portion of the hospital. The magnitude and duration of the impact, as well as the effect upon normal activities, may vary greatly. Involvement with an adverse event, particularly a preventable or never event that involves significant injury or death, can be traumatic and may cause a grief event for the staff member.

As used within this disclosure, the phrase “break status” identifies whether the staff member is on a break, i.e., temporarily not actively providing care or providing care at a reduced level. Given the long shifts often worked by staff members and the nature of the care being provided, it is important for staff members to periodically have a break. This may be indicated by the presence in a particular location, e.g., in a lunchroom (having a meal). This may also be indicated by a staff member actively indicating that they are taking a break, e.g., by changing their status on a physical tracking board or in a software program. In certain embodiments, it is desirable to avoid interrupting a break of the otherwise first-choice recipient, due to the adverse impact on that staff member, when another staff member can handle an alarm.

As used within this disclosure, the term “back-up” identifies a staff member who has been determined to have the ability to provide at least a portion of the care normally provided by another staff member. There is an expectation that the back-up will be called on only infrequently, as the back-up will have their own responsibilities. Certain back-ups, e.g., a peer or a supervisor, may be identified as a back-up for all responsibilities of a staff member. Certain back-ups may be identified as a back-up only for specific types of alarms, e.g., a technician being a back-up to a nurse only for a low battery alarm.

### Do not Disturb Status

At times, a staff member may be provided with an alert that is associated with an alarm or condition that requires an immediate response and cannot be paused to take care of a second alarm. In certain embodiments, the system and method disclosed herein assigns a Do Not Disturb (DND) status to that staff member. The DND status persists, i.e., is “active,” until it is reported that the alarm has been satisfactorily resolved. In this context, certain conditions, e.g., fire, a disaster, or severe weather, are considered interchangeable with an alarm as far as initiating an alert and are considered active until the condition is no longer present.

The system assigns a DND status based on an evaluation of one or more dynamic attributes of alarms for which an associated alert was sent to the staff member. In certain embodiments, the dynamic attributes are included in a group that comprises the number of active alarms that have a sub-priority greater than a predetermined level, the number of active alarms that require a response having a complexity equal to or greater than a predetermined level, and the number of emergency codes. In certain embodiments, the system will not send an alert for other alarms to a staff member that has an active DND status.

### Emergency Codes

Emergency codes and associated colors are defined by a health care facility to coordinate a response by staff members to situations that require immediate action. A code is declared when the event occurs. While the definition of which code color is associated with an event is not universal, exemplary guidance defines the following colors:

TABLE 3

| EMERGENCY | |
|---|---|
| CODE | MEANING |
| Red | fire (seeing flames, seeing or smelling smoke) |
| Blue | adult emergency, such as cardiac arrest or |
| | respiratory failure |
| Yellow | bomb threat; disaster |
| Gray | combative person; severe weather; security |
| | response required |
| Silver | a weapon or hostage |
| Pink | infant or child abduction |
| Orange | hazardous material accident |

An emergency code may be declared and publicly announced, for example over a public address (PA) system or broadcast electronically to multiple staff members, e.g., everyone in a building, via their personal devices, e.g., mobile phones. These actions are considered as equivalent to an alarm being issued by a medical device, an assignment of the highest priority, and provision of an associated alert to a staff member. In certain embodiments, the dynamic attributes include a location at which an emergency code has been declared and a location of the staff member and the DND status is assigned to the staff member if the staff member is within a predetermined distance of the emergency code location.

### Tasks and Breaks

Breaks are an essential aspect of working in a care facility or other high-criticality position. It is necessary to have some “down time” to rest when the routine activities require careful attention. In certain embodiments, a break comprises one or more of (a) a period of time where a staff member is not expected to perform routine, scheduled or any tasks, (b) a meal break, (c) leaving a shift early, (d) attending a training session, a counseling session, or a meeting with management or other stakeholders, (e) a grief period, (f) a period of time where a staff member is expected to sleep at or outside the facility, or (g) a period of time where non-routine tasks are performed, e.g., time to finish documentation tasks. Taking the full number and duration of scheduled breaks improves both a staff member's performance and the well-being of the staff member. Many staff members are dedicated to their jobs and reluctant to take time away unless they are certain that their responsibilities are being covered by another staff member that they trust. For example, hospital nurses are known to often skip their scheduled breaks because there is nobody assigned to take care of their patients if something happens while they are on break or they feel guilty about giving their workload to another staff member who is already overburdened with their own high workload. The responsibilities of a nurse are constantly changing, dependent upon the conditions of their current patients, and manually managing coverage so a nurse can go on break is a difficult and time-consuming task.

An automated system configured to evaluate the current workload of a nurse and determine whether assigned tasks can be “paused” without impact on the patients assures the nurse that patient care will not be compromised while the nurse takes a break. The disclosed system also provides the ability to re-assign tasks to enable a staff member to go on a break. The disclosed system also analyzes and modifies dynamic attributes, e.g. break and staff dynamic attributes, so breaks can be optimally scheduled, and more breaks can be executed on time and without interruption. Interruption of breaks is widespread and leads to clinician fatigue.

In addition, breaks laws have recently been passed in the United States, specifically the states of California, Oregon, and Washington. These states have moved to hire dedicated break nurses to cover the breaks of nurses. However, these break nurses lack a system that sends them prioritized tasks for them to complete during the break periods they cover for other clinicians. They also lack a system that optimizes break scheduling, break execution, and task reporting to ensure optimal break coverage. This system allows them to break more staff members concurrently and work shorter shifts thus saving valuable resources for hospitals. Furthermore, hiring nurses to specifically cover breaks exacerbates the nurse shortage issue in many regions.

The Task and Break Management system integrates other dedicated support staff, e.g. certified nurse assistants, and reassigns a lower-level task from nurses to support staff or escalates back up to nurses if tasks are not completed in certain time.

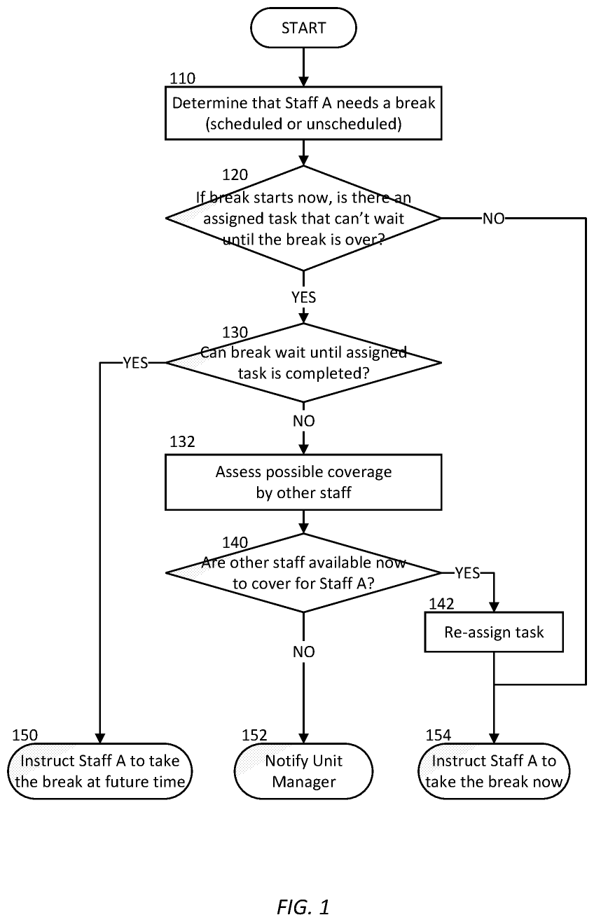

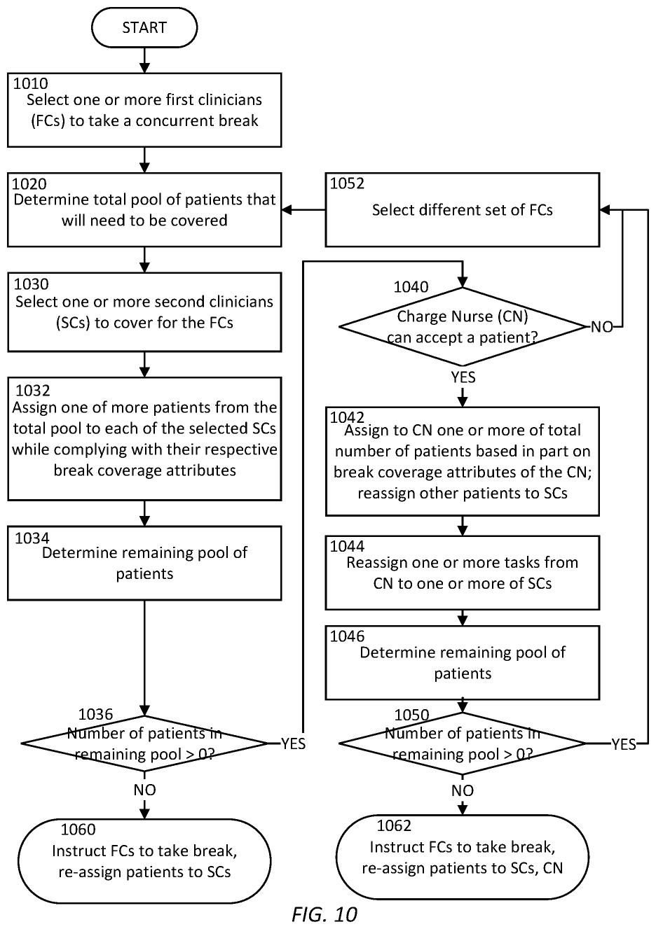

FIG. 1 depicts an exemplary workflow for managing a break for an individual, according to certain aspects of the present disclosure. The process starts at step 110 where the system determines that a staff member, designated in this example as Staff A, needs a break. In certain embodiments, a break can be a scheduled break that is retrieved from a break schedule, e.g., from a Staff Management system 1016 as shown in FIG. 10. In certain embodiments, a break is requested by Staff A for themselves, by a manager for Staff A, or by another coworker for Staff A. In certain embodiments, the system determines an unscheduled break based on one or more of hospital policies, local, state, or national compliance requirements, or best practices to combat fatigue and sleep debt. In certain embodiments, the system retrieves the staff dynamic attributes of a staff member and determines that a break is needed. In certain embodiments, the system reviews attributes associated with workload and fatigue and determines that a break is needed because a staff member has worked too many hours in a specific time period. In certain embodiments, the system reviews attributes associated with workload and fatigue and determines whether an unscheduled break is needed based on rules to reduce the risk of an adverse event. In certain embodiments, a break comprises a reduction of tasks assigned to a staff member for a period of time. For example, a staff member may receive a break to finish documentation for their shift but still be eligible to be assigned a particular type of task, e.g., an ad hoc task to respond to an alarm, while on this “documentation break.”

Step 120 determines whether Staff A has an uncompleted assigned task that cannot wait to be completed until a break that starts now is over. In certain embodiments, there is a task list associated with Staff A and each task has one or more attributes, e.g., the task must be completed by a defined time or the task has an estimated time-to-complete duration. The system determines whether a task can be deferred until after the determined break by comparing the expected task durations with the break start time and duration. In certain embodiments, the system determines whether the task can be deferred by the priority assigned to the break. In certain embodiments, only critical tasks, e.g., tasks that have been assigned a priority above a threshold, are considered to determine if Staff A can take a break. In certain embodiment, some tasks may be defined or configurable as not to be reassigned based on hospital, unit, or staff policies or preferences. For example, some nurses prefer to perform their own medication titrations.

In certain embodiments, the list of tasks assigned to a staff member pulls tasks associated with the patients assigned to the staff member from various sources, e.g., a medical device 1022, a pharmacy system 1014, an electronic health records (EHR) 1010 of FIG. 10. In certain embodiments, tasks are manually assigned to staff members, i.e., added to the task lists of the staff members.

If step 120 determines there are no tasks that need to be completed during the break period, the process branches to step 154 that instructs Staff A to take the break now. In certain embodiments, the break starts immediately upon this instruction. In certain embodiments, the system instructs Staff A when to start the break. In certain embodiments, the system provides an advanced notification to Staff A that their break will start in X minutes in step 154 prior to performing the task analysis of step 120. For example, the advanced notification alerts the clinician that their break starts in 10 minutes and determines if the staff member should finish any tasks before they take the break.

If step 120 determines there are tasks that need to be completed during the break period or during the advanced notification window, then the process continues to step 130 where the system determines if the break start time can be delayed until the assigned task is complete. In certain embodiments, a variable time-based critical task (e.g. a nurse performing a drug titration, working a code, etc.) does not allow a clinician to take a break during the defined start time. In certain embodiments, a clinician may enter an input to indicate they cannot take a break at the scheduled start time. For example, they may decline the break notification task on their mobile phone, or state they cannot take a break via a voice activated device. In other embodiments, if the clinician does not accept a break during an advance notification window and the system knows there is a critical task on their task list, then the system will not start the break and reassess the revised break start time at a later time.

If step 130 determines the break can wait until an assigned task is completed, then the process branches to step 150 and instructs Staff A to take a break at a future time that allows enough time to complete the conflicting task. In certain embodiments, the system provides a notification that Staff A may start their break within a range of times in the future. In certain embodiments, the staff member's manager may designate that the break needs to be taken at the defined start time. In certain embodiments, the rules covering when breaks need to start are predefined in configurable settings and rules, e.g., defined by a hospital administrator in the Task and Break Management system 1040 of FIG. 10.

If step 130 determines the break cannot wait until the task is completed, then the process branches to step 132 that assesses coverage by other staff members. In certain embodiments, the system retrieves the staff dynamic attributes of other staff members and determines who is qualified to perform the task. In certain embodiments, the system retrieves the workload or task list of other staff members and determines who has the bandwidth to perform the task within the required time window. The process then continues to step 140.

Step 140 determines whether there is an available staff member to whom the task can be reassigned. In certain embodiments, the system retrieves staff dynamic attributes of the other staff members on the unit and determines who should be reassigned the task. In certain embodiments, the system determines whether there are dedicated resources who can perform the task, e.g., a break nurse, or if the other staff members working the unit can perform the task. If there are staff members available to perform the task, the process branches to step 142 wherein the task is reassigned and then step 154 instructs Staff A to take the break.

If step 140 determines there are no available staff members to whom the task can be reassigned, the process branches to step 152 wherein the unit manager is notified that the task could not be reassigned and the break could not be deferred such that Staff A was not able to take their assigned break. In some embodiments, the system logs these notifications and provides a signal if these notifications exceed a threshold. In some embodiments, the system provides managers with configurable options on how they want to be notified.

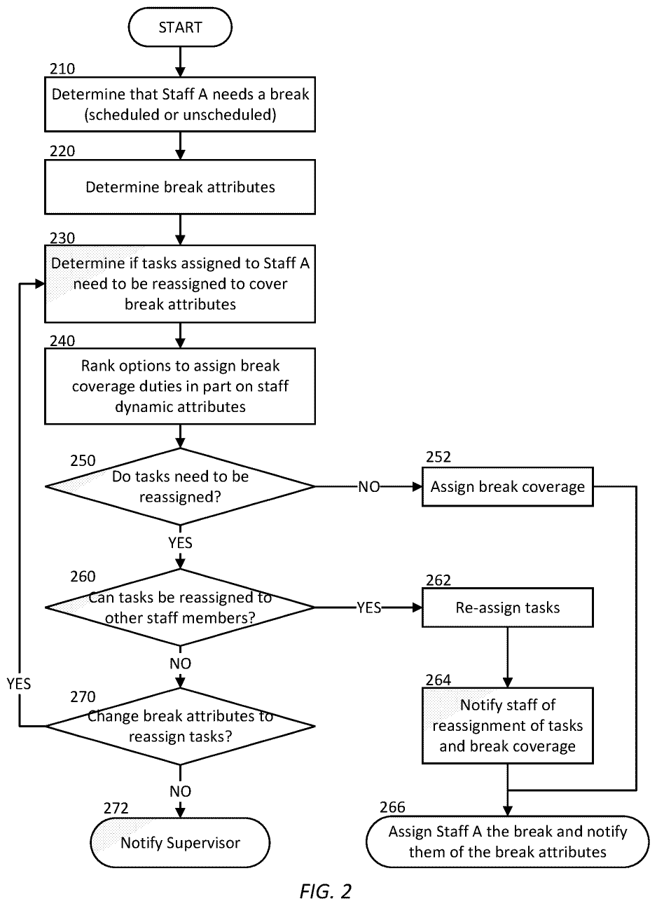

FIG. 2 depicts an exemplary alternate workflow for managing options for giving a staff member a break, according to certain aspects of the present disclosure. The process starts at step 210 wherein the system determines that Staff A needs a break.

The process continues to step 220 where the Task and Break Management system determines the dynamic attributes of the break. In certain embodiments, the break dynamic attributes comprises one or more of a start time, a duration, a break type, e.g., a meal break or a training session, a break priority, a second staff member to share the break with, a location of the break, an item to bring to break, e.g., training material, and what type of tasks to reduce and what type of tasks to still assign. In a first example, the system determines the attributes of a break for Staff A comprise that this is a lunch break, the duration is 60 minutes, the break starts in 15 minutes, and Staff B is to share this break with Staff A. In a second example, the system determines the break dynamic attributes comprise that this is a 1-hour training session at 2 PM in Training Room A, and Staff A is to bring training material. In a third example, Staff A is not feeling well, and the break duration attribute needs to account for the last six hours of their shift.

At step 230, the Task and Break Management system determines whether any tasks assigned to Staff A need to be reassigned to other staff members to execute the break dynamic attributes. The process proceeds to step 240 where the system determines options to reassign tasks, if any tasks need to be reassigned, and ranks them. In certain embodiments, the system determines if tasks can be reassigned to other staff members per their staff dynamic attributes. For example, the system determines if other staff members are available and capable of performing the tasks and do not have a workload status, burnout status or performance status that prohibits them from taking on additional tasks. For example, the system will not reassign the task to a particular staff member if it increases the probability of an adverse event above a threshold. In certain embodiments the system prioritizes reassigning tasks to the lowest level of staff that is trained to perform the task.

In certain embodiments, the system will only provide options that reassign certain tasks but not others. In certain embodiments, only critical tasks or tasks associated with the admit, discharge, and transfer (ADT) goals of the facility are reassigned to other staff members. In certain embodiments, tasks are categorized in the Task and Break Management system, e.g., a list of tasks that cannot be reassigned. In certain embodiments, the system maximizes the number of staff members that a dedicated break nurse covers within a time period by identifying options where some tasks are assigned to the break nurse and other tasks are assigned to other staff members, e.g., a technician. In certain embodiments, an option that enables a break nurse to cover for more staff members while limiting reassignment of tasks to other staff members with assigned patients are ranked highest.

In certain embodiments, the system comprises rules to ensure compliance with regulations. For example, some state regulations specify maximum pre-defined patient/staff ratios. Some regulations allow patient tasks to be split between one or more staff members to ensure flexibility in interpreting the maximum allowed patient/staff workloads.

In certain embodiments, the system will consider staff members who are not currently working the shift and determine options that best consider the staff dynamic attributes of these staff to reassign the tasks. For example, these other staff members may be “float staff,” work in a different unit, on-call, assigned to work the next shift, remote, and from staffing agencies. In certain embodiments, the system will first prompt the supervisors to call in the additional resources before reassigning tasks.

In step 250, the system determines if, based on availability of additional staff, there are still tasks be reassigned to other staff members. If tasks must be reassigned, the process branches to step 252 that reassigns the tasks then progresses to step 266 wherein the system assigns Staff A the break. In certain embodiments, these staff members receive a communication that notifies them that an imminent critical task has been reassigned to them and they need to perform the task immediately. In certain embodiments, the staff receive a notification that new tasks have been reassigned and the tasks are in their task assignment list. In certain embodiments, the assigned and reassigned tasks will appear in a task management list that is located on a mobile phone application, a desktop application, a digital whiteboard, or a digital screen located in a patient room, a hallway or a central location.

In certain embodiments, step 250 determines that there are no current tasks that need to be reassigned but makes a determination that potential ad hoc tasks may occur during the break period and branches to step 252.

In certain embodiments, the assignment of the break prevents other tasks from being assigned to Staff A during the break duration. In certain embodiments, ongoing tasks can still be assigned to Staff A during their break as long as the tasks may be performed after the break is over. In certain embodiments, the system notifies Staff A to whom their critical tasks were reassigned to and acknowledgment that the other staff member accepted the reassigned task. For example, if Staff A requested a 20 min break to occur immediately and they had to administer a critical medication ten minutes into such break, the system will notify them that Staff C accepted the assignment to perform the critical medication administration. In certain embodiments, Staff A receives another notification that the critical medication was administered.

If step 250 determines that at least one task needs to be reassigned to another staff member, the process branches to step 260 wherein the system determines which staff members are qualified and available to accept each task. If the task can be reassigned, the process branches to step 262 that reassigns the task, step 264 that notifies the staff members receiving the reassigned tasks, and step 266 that notifies staff A to take the break.

If there are tasks that cannot be reassigned in step 260, the process branches to step 270, which evaluates possible changes in the break dynamic attributes, e.g., start time. If an attribute can be changed, the process branches back to step 230 to reevaluate what task need to be reassigned.

If step 260 determines the break dynamic attributes cannot be modified and, therefore, it is not possible to reassign tasks to cover the initial break dynamic attributes, then the process proceeds to step 262 and notifies the supervisor.

In certain embodiments, the system provides the supervisor the best options to provide the break to Staff A, the break dynamic attributes for this period, and what tasks cannot be assigned to other staff members. In certain embodiments, the system asks the supervisor if they can personally cover the one or more tasks that cannot be covered by the other staff and, if so, the system will reassign the one or more tasks and notify the parties (steps 252, 262, and 266).

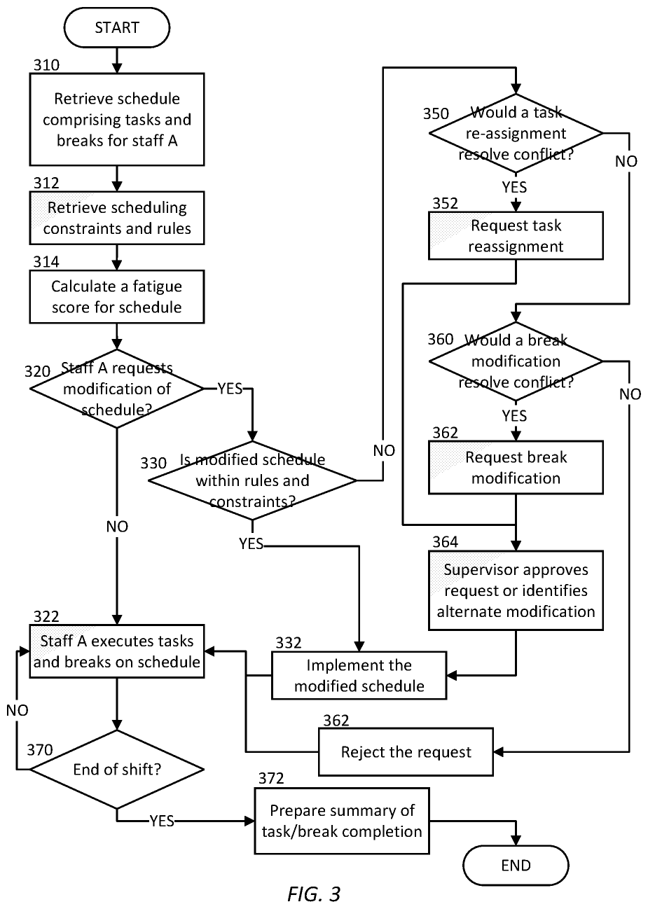

FIG. 3 depicts an exemplary workflow for enabling a staff member to control their own break schedule, according to certain aspects of the present disclosure. Steps 310, 312, 314 retrieve one or more of assigned tasks, assigned breaks, and a schedule for staff A and calculates a fatigue score for this schedule for staff A. In certain embodiments, the fatigue score is calculated from one or more of hours worked in the most recent time period of a defined length, the number of hours worked continuously immediately prior to the current time, the time since a last break, the number of patients currently assigned to the staff member, the number and/or complexity of currently assigned tasks, an assessment by a supervisor or other staff member, and results of a questionnaire/assessment document.

In step 320, Staff A requests a break at a proposed start time with a proposed duration. In certain embodiments, the request comprises one or more of changing the current break start time to a proposed start time, changing the break duration to a proposed duration, and ending a current work shift at a proposed time.

Step 330 determines whether the requested break complies with rules and constraints defined in the system and, if compliant, implements the requested change in step 322. If the requested change is not compliant, e.g., a task would not be completed within the allowed time window, the process branches to step 350 that determines whether a task reassignment will resolve the conflict. If a reassignment is identified that will resolve the conflict, the reassignment is requested in step 352 and presented to the supervisor, who approves the reassignment or identifies an alternate modification in step 364. If step 350 cannot identify a task reassignment that resolves the conflict, the process branches to step 360 to determine a break modification that would resolve the conflict. If a modification is identified, the modification is requested in step 362 and again forwarded to the supervisor for review in step 364. If there is no break modification that will resolve the conflict, the system rejects the requested schedule modification in step 362.

In the absence of an action by staff A in step 320, the process proceeds to step 322 wherein staff A executes the schedule, performs the assigned tasks, and takes the assigned breaks until the end of shift in step 370. In certain embodiments, step 372 prepares a summary of tasks and breaks that includes whether the tasks were completed and the breaks taken.

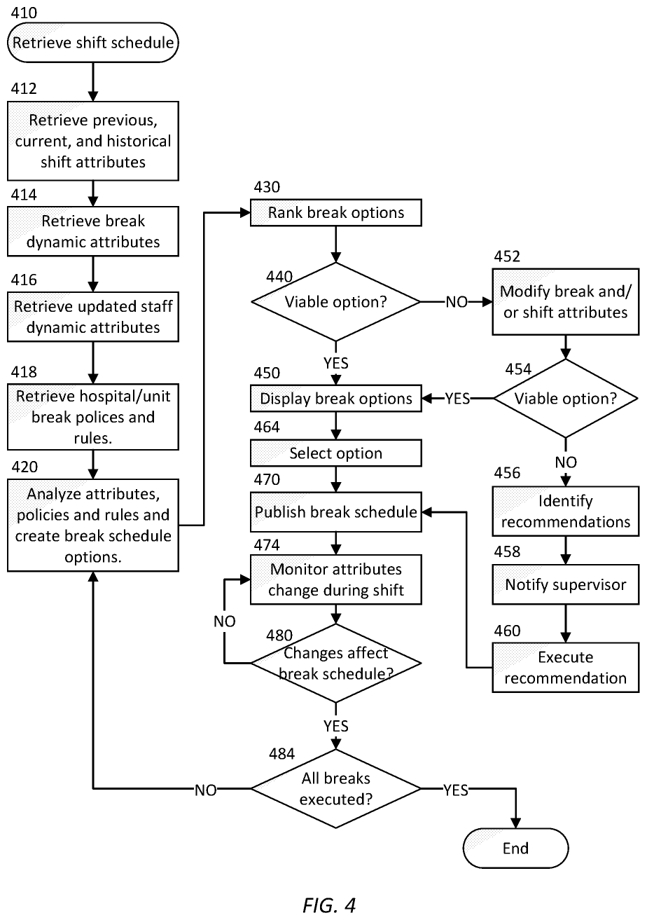

FIG. 4 depicts an exemplary workflow for managing a break schedule for staff members in a care unit, according to certain aspects of the present disclosure. The Task and Break Management system retrieves the shift schedule for the shift that the break schedule needs to be created, shifts attributes from one or more of the previous, current, and historical shifts, break dynamic attributes, staff dynamic attributes, and policies and rules in steps 412, 414, 416, 418. In certain embodiments, the shift schedule can be imported from a staff management system, e.g., a scheduling program. In other embodiments, the shift schedule is manually entered by a staff member. Shift dynamic attributes are defined as attributes that can change on a shift or between one shift and another and include one or more of staffing, shift hours, type of shift (e.g., day shift or night shift), ADT information, type and severity of medical conditions treated, patient/staff assignments ratios, acuity of patients, stability of patients, number of overtime (OT) hours scheduled and/or worked by staff on a unit, availability of support staff (e.g., certified nursing assistants and/or break nurses), number of breaks to be scheduled, breaks hour to be scheduled, number of breaks missed or interrupted on a prior shift, community events near hospital during shift, and weather and traffic events during shift. In certain embodiments, shift dynamic attributes include outstanding tasks that need to be carried over from the previous shift to the shift being planned. Break dynamic attributes comprises one or more of break type, duration, priority, break start time, number of breaks per clinician per shift, and staff break preferences. In certain embodiments, staff dynamic attributes comprise an item in Table 2 or one or more of staff who are working the current shift, staff who worked the previous shift and who were asked to continue working the current shift, and staff who are currently not working or are working at different units, e.g. float nurses, that can be called in to work the shift being planned.

The method proceeds to step 420, where the Task and Break Management system analyzes the shift, break and staff dynamic attributes, break policies and rules, and staff break preferences and creates different break schedule options. In certain embodiments, the various attributes are weighted. The process proceeds to step 430 wherein the system ranks the different options. In certain embodiments, the system will prioritize rules and analyze the weights of the applicable dynamic attributes associated with the prioritized rules to create higher-ranking break schedule options. In other embodiments, there are hard constraints that may not be violated. For example, meal breaks can be as long as one hour but cannot be less than 30 minutes per state regulations. In certain embodiments, the combination of hard rules and weighted scores for dynamic attributes will determine ranking for break schedule options.

Step 440 will determine if there is a viable option among the different ranked break schedule options. In some embodiments, some options are not viable because the break management system is unable to schedule all the breaks or schedule all the breaks with the required break dynamic attributes. For example, inadequately staffed shifts with high patient workloads will not have a viable break schedule option because not everyone will be able to take their breaks per their break dynamic attributes.

If there is a viable break option, then the break management system will proceed to step 450. In step 450, the break management system will display the break schedule option rankings to managers and administrators. In certain embodiments, each break schedule option is ranked with a score between 0 to 100, with a scoring system that defines different scores ranges by acceptability and indicates the acceptability visually on a user interface. For example, from 0 to 100, any score less than 50 is not acceptable and the break management system will display the score with red font, an unacceptable designation, or both. In other embodiments, the break schedule options with the highest ranking are shown on the top of the user interface and the lower ranked options are shown at the bottom of the user interface or not at all. In other embodiments, the system will generate a break management schedule and indicate which breaks have a low confidence of occurring.

In step 464, the manager or the break management system selects the break schedule option to publish for the unit. In certain embodiments, the Task and Break Management system selects an option and proceeds to step 470. In other embodiments, a manager selects the option or approves the selected option at step 464 and then the system will publish the schedule at step 470. In certain embodiments, the manager approves a partial break schedule where one or more clinician breaks need to be filled. In certain embodiments, the manager approves a partial shift break schedule, e.g., just the morning breaks. In certain embodiments, the break schedule is published on one or more display devices in the unit, e.g., mobile phones, desktop applications, and/or digital whiteboards.

If there is no viable break schedule option in step 440, the process branches to step 452 and modifies one or more dynamic attributes, e.g. a break dynamic attribute or a shift dynamic attribute. In certain embodiments, the first attempt at creating a break schedule option will consider the maximum value of a break dynamic attribute. In an example wherein regulations require a meal break to be at least 30 minutes in duration and hospital policy is to provide a 60-minute meal break, then step 452 decreases the meal break duration, with a minimum of 30 minutes, to identify a viable break schedule option.

In certain embodiments, the initial break schedule creation will not consider certain shift dynamic attributes, e.g., the use of a dedicated break nurse. If this constraint does not identify a viable option, then step 452 will add a dedicated break staff member and re-evaluate the options. This method allows a unit manager to determine how long they may need to keep a dedicated resource to cover the breaks of the shifts.

Step 454 will determine if there is a viable option after the modifications. If yes, then the method will proceed to step 450 and display the break options. If no, then the Task and Break Management system will identify recommendations in step 456 to create a viable break schedule. For example, a recommendation may include advising a charge nurse to cover breaks for 30 minutes during the morning breaks, recommending a dedicated break nurse be used from 11:00 AM to 2:00 PM, or calling in a staff member who works the next shift to come in one hour early. In step 458, the break management system will notify the supervisor that there are not viable break schedule options, provide the reason, and display the identified recommendations.

The supervisor can execute one of the recommendations in step 460. In certain embodiments, the user interface may provide options to select on the screen which will implement the recommendation. Once the recommendation is implemented then the break management system proceeds to step 470 and publishes the break schedule. In other embodiments, the recommendation cannot be executed completely by the Task and Break Management system. For example, the recommendation may be to bring in extra staff members but it takes time for the extra staff member to clock in and, in certain embodiments, the break management system publishes the break schedule in step 470 with color codes indicating breaks that can be taken without the recommendation and breaks that be taken with the recommendation. In certain embodiments, the recommendation is displayed in the schedule with the applicable color coding.

In certain embodiments, the Task and Break Management system will continuously monitor the dynamic attributes in step 474 and update the break schedule if warranted. In certain embodiments, the system will monitor the staff, shift, patient and break dynamic attributes. For example, if the fatigue score changes for a staff dynamic attribute, the break priority will change for this clinician, and the break management system will look to schedule this break sooner. In another example, staff members are added or subtracted to a unit and the applicable shift dynamic attribute changes causing the break management system to modify the break schedules or identify recommendations for the manager. In certain embodiments, a clinician requests a break and the system accommodates the break if the manager approves the request.

When a system detects a shift, break, patient or staff dynamic attribute change, step 480 determines if the change is significant enough to affect the break schedule. In certain embodiments, there is a subset of attributes that can affect break schedules and there are thresholds that determine if the attribute change is significant enough to affect a break schedule. In certain embodiments, the determination for whether an attribute change is significant enough depends on how much time is remaining on the shift, if breaks have been executed or if there have been missed/interrupted breaks that need to be rescheduled. For example, if there is one hour left in the shift and there are only a few 15-minute breaks to execute on, then the system may determine most changes are not significant compared to if the evaluation was made earlier in the shift.

If step 480 determines the attributes changes do not affect the break schedule, then the system returns to step 474 and continues monitoring for attribute changes. In certain embodiments, the dynamic shift and/or break dynamic attributes change as staff members enter information into the system for events, e.g. break completion. If the break schedule is affected, then the system branches to step 484 and determines whether all the breaks have been executed for the shift. If step 484 determines that all the breaks have been taken on the shift, the process ends. If some breaks have not yet been completed, then the process returns to step 420 and reanalyzes the break schedule.

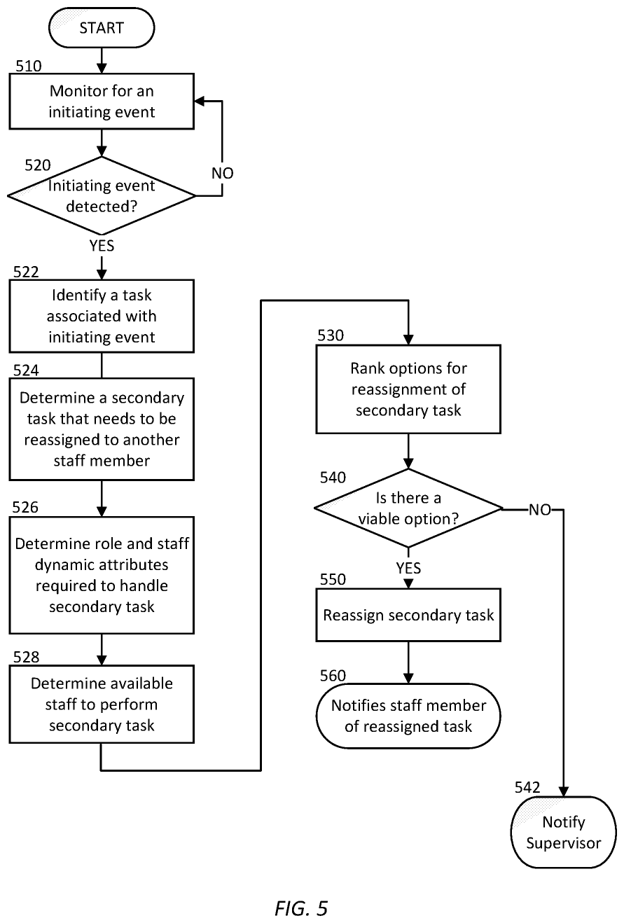

FIG. 5 depicts an exemplary workflow for assigning a task in response to an initiating event, according to certain aspects of the present disclosure. As used in this disclosure, the phrase “initiating event” refers an event where one or more clinicians cannot take any tasks (e.g., a clinician taking a break, going home early, attending a training session etc.) or an event where one or more clinicians can only take tasks from one patient over set amount of time (e.g., working a code, patient-sitting a patient who requires special 1:1 care per hospital policy) when they normally are assigned more than one patient. An initiating event requires that the tasks associated with all the patients assigned to a clinician or the tasks associated with all the patients except one patient be reassigned to another clinician since the primary clinician is not available to perform these tasks.

The process starts at step 510, where the Task and Break Management system monitors for the presence of an initiating event. In certain embodiments, the application receives a notification of an initiating event from another hospital system via a communication protocol. For example, a nurse call station can communicate a code blue event. In certain embodiments, a clinician, remote clinician, or a staff member can enter an initiating event into the Task and Break Management system. For example, a charge nurse can create a task that requires a clinician to patient sit a patient for the next hour. In certain embodiments, the Task and Break Management system determines a break schedule for a staff member is scheduled in 15 minutes and needs to be executed.

The process proceeds to Step 520 where the Task and Break Management system determines if the input received qualifies for an initiating event. In certain embodiments, the system allows the hospital to create rules to define an initiating event. For example, an administrator creates a rule that consider the attributes of the event, e.g., duration and severity, patient dynamic attributes of the patients assigned to a clinician, e.g., patient acuity, and staff dynamic attributes of the clinician, e.g., patient assignment load. The administrator can define rules so an initiating event is detected if Clinician A has to address a code but has a high workload, e.g., other patients assigned, with high patient acuities.

If an initiating event is not detected, then step 520 returns to step 510 and continues to monitor. If an initiating event is detected, then the process proceeds to step 522 and identifies the secondary tasks associated with the initiating event. As used in this this disclosure, the phrase “secondary task” is a task not associated with the initiating event but are associated with the tasks that the clinician cannot perform when they are performing the initiating event. For example, when a clinician is on break or working a code, they may have secondary tasks associated with other patients assigned to them that require care, e.g., medication administration or vital sign monitoring.

Step 524 determines which secondary tasks should be reassigned to another staff member. In certain embodiments, the system determines which tasks need to be reassigned based in part on a patient dynamic attribute associated with the tasks, e.g., patient acuity. In certain embodiments, the tasks that need to be reassigned based on the task priority and attributes of the initiating event, e.g., duration. For example, high and medium priority tasks that need to be completed before the initiating event ends would be reassigned. In certain embodiments, a break nurse who is covering breaks for multiple clinicians receives tasks reassigned that are prioritized based on the dynamic attributes from multiple initiating events.

The process proceeds to step 526 and determines the role and staff dynamic attributes required to handle the secondary tasks. In certain embodiments, the Task and Break Management system determines the training required to perform a task and will consider the type of medical device or therapeutic, the type of procedure and if it is specific to the hospital or the hospital unit, and any new safety information from the manufacturer, e.g., new way to perform a task on a device based on a recall. If the staff member does not have the proper training, the Task and Break Management system will not reassign the secondary task to that staff member.

Step 528 determines who is available to perform the tasks. In certain embodiments, a clinician on break is not available. In certain embodiments, a staff member who is on one initiating event, e.g., break, may need to be summoned back to perform secondary tasks for a clinician on another initiative event, e.g., participating in a code. Step 530 ranks the options for reassignment of secondary tasks. In certain embodiments, the system ranks staff members based on one or more of their staff dynamic attributes. For example, the Task and Break Management system determines what training is required to perform a task and will not assign a task to a new staff or float member who is not trained on the task. In certain embodiments, the patient dynamic attribute and the staff dynamic attribute are considered. For example, Staff A may lack the training but, due to their education and the staff dynamic attributes of the other staff and the acuity of the patient associated with the task, the task is still reassigned to Staff A.

Step 540 determines if there is a viable option and, if no, then step 542 notifies the supervisor. In certain embodiments, the system notifies the supervisor of the initiating event, the secondary tasks that cannot be reassigned, the task priority and information to identify the patient and patient location.

If step 540 determines there is a viable option, then step 550 reassigns the secondary tasks. The process ends with step 560 and notifies the staff members of the reassigned tasks. In certain embodiments, the staff member who is receiving the reassigned task receives the assignment on a notification device (e.g., mobile phone, voice activated device, central workstation). In certain embodiments, the receiving clinician's task list is updated to reflect the reassign secondary tasks with the reassigned task, task priority, applicable notes and information to identify the patient, patient location, and the staff member who was originally assigned the task. In certain embodiments, the staff member who is performing the initiating event also receives a notification. For example, they receive a message on a display device or their task list is updated to state the status of the secondary task, who performed it, the time they performed it, and any applicable notes.

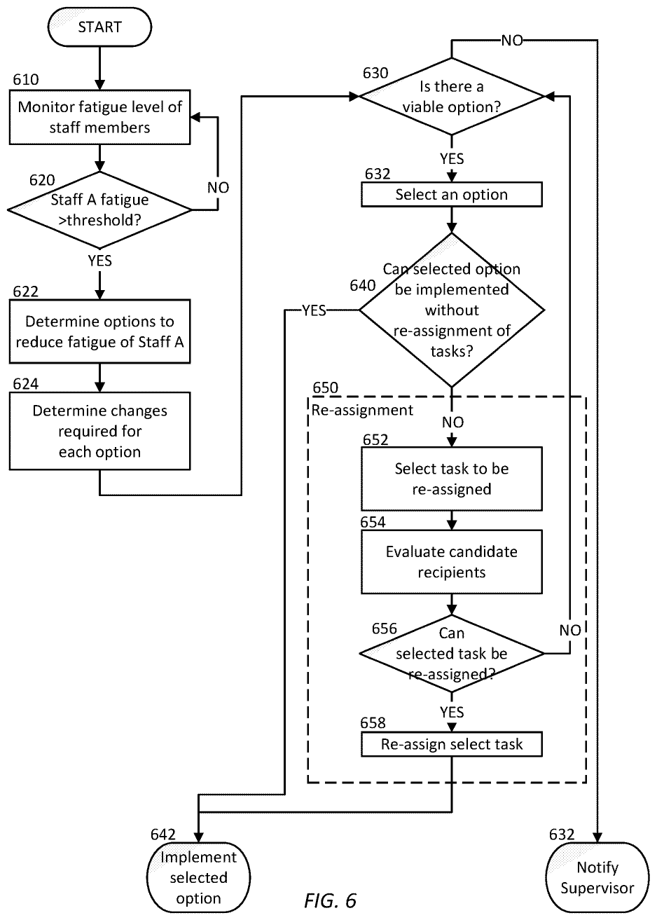

FIG. 6 depicts an exemplary workflow for reducing fatigue in a staff member, according to certain aspects of the present disclosure. The process starts with step 610 that monitors the fatigue level of the staff members, e.g., using a self-administered questionnaire or a supervisor assessment, that is compared to a threshold in step 620. In some embodiments, monitoring comprises analyzing the work schedule of the staff member to understand how many shifts and hours they have worked in a given time period. In certain embodiments, monitoring the burnout or fatigue staff dynamic attribute of a staff member and determining if a threshold is exceeded determines if staff fatigue should be mitigated.

If a staff member is found to be fatigues in step 620, the process proceeds to determine options to reduce that staff member's level of fatigue, determine what changes are required for each option, and determine whether there is a viable option in steps 622, 624, and 630. If there is no viable option, the process notifies the supervisor in step 632. If there is at least one viable option, the process proceeds to step 632 and selects one option to evaluate in step 640 as whether that option requires reassignment of a task. If a reassignment is not required, the selected option is implemented in step 642. If a reassignment is required, the process proceeds to block 650 wherein steps 652, 654 select a candidate task and determine whether the candidate task can be reassigned. If the candidate task can be reassigned, the process reassigns the task in step 658 and implements the selected option in step 642. If the candidate task cannot be reassigned, the process loops back to step 630 reconsider the options. In certain embodiments the process branches first from step 656 to step 652 to select other candidate tasks and only branches back to step 630 if no task is found to enable the selected option.

In certain embodiments, options to reduce fatigue comprise one or more of reassigning a task, reducing certain types of tasks, reducing distance between tasks, e.g., reducing the walking, sending the staff member on a break, modifying the break attribute to give them a longer break or a sleep break, sending the staff member home early, and reducing the number of patients assigned to the staff member.

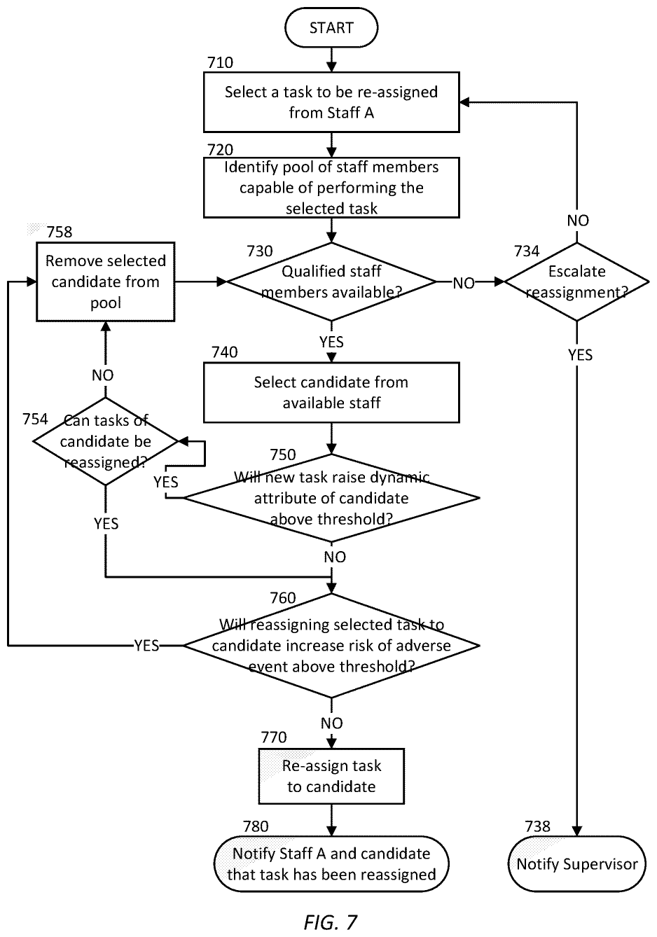

FIG. 7 depicts an exemplary workflow for re-assigning a task from one staff member to another staff member, according to certain aspects of the present disclosure. This example presumes that a task must be reassigned from Staff A to another staff member. The process starts at step 710 when the Task and Break Management system selects a task to be reassigned from Staff A. For the purpose of this example, the method of selection are not important. In step 720, the system determines a pool of staff members capable of executing the selected task. In certain embodiments, each task is prioritized by severity and level of skill, e.g. education and/or experience, required to perform the task and the system determines which staff have the requisite staff dynamic attributes. For example, certain medications may only be administered by a nurse or a doctor. From this pool of capable staff members, step 730 determines which staff members are available to perform the task. In certain embodiments, availability is based on one or more of whether they are currently working, whether they are on a break, whether they are on a DND status, or whether they are located at a distance that prohibits them from responding in a timely or reasonably fashion. In certain embodiments, the location of the staff member is determined by a Real Time Locating System (RTLS) that tracks the staff member, e.g., by sensing the location of one or more of the staff member's badge and their personal communication device, or a passive system, e.g., facial recognition from surveillance cameras that are static or attached to a moving platform within the hospital.

If step 730 determines a qualified staff member is not available for the task that requires reassignment, then the process proceeds to step 734 to determine if the escalation time period for reassignment has been escalated. If yes, then the system will notify the supervisor in step 738. If no, then the system will continue to step 710 and continue to cycle through steps 710, 720, 730 to find an available qualified staff member or step 734 determines the reassignment needs to be escalated.

If step 730 determines one or more qualified staff members are available, then step 740 will select a candidate. In certain embodiments, the system preferentially selects a staff member pre-assigned to support Staff A, e.g., a “buddy nurse.” In certain embodiments, the system ranks the available staff members by one or more of their staff dynamic attributes and initially selects the highest ranked staff member. In some embodiments, the dynamic attribute score used to rank the best candidates is a weighted average of the individual scores of the staff dynamic attributes.

Step 750 determines if reassigning this new task or set of tasks to the candidate will cause one or more of the candidate's dynamic attribute score to exceed a threshold. If no, then step 760 determines if reassigning the task to the candidate will cause the risk of an adverse event to increase above a threshold. In certain embodiments, the risk of an adverse event is based in part on one or more of a fatigue level, the role of the staff member, the tasks that are currently assigned to the staff member, a dynamic attribute of a patient assigned to the staff member, and a dynamic attribute of the staff member. If no, then step 770 will reassign the task or set of tasks from Staff A to the candidate and notify both Staff A and the candidate that the task has been reassigned from in step 780. In certain embodiments, steps 750 or 760 is omitted from the process. For example, a break nurse may be reassigned tasks from a physician going on break.

If step 750 determines the reassignment of the task will increase the candidate's dynamic attribute score above a threshold, then step 754 will determine if a task originally assigned to the candidate can be reassigned to another staff member so the candidates dynamic attribute score will stay below the threshold. In certain embodiments, the selection of which tasks from the candidate is accomplished using the process of FIG. 7. If tasks cannot be removed from the candidate's task list to accommodate reassignment of the task from Staff A, then step 758 removes Staff B from the candidate pool then proceeds to step 730 to determine if there is another staff that is qualified. In certain embodiments, there are timers built in for how long a task can remain not completed before escalating to another staff member or a unit manager. In certain embodiments, a high sub-priority task assigned to Staff A is reassigned to the unit manager if no qualified staff members are identified.

If step 754 determines tasks can be reassigned from Staff B, then the process proceeds to step 760 and determines if reassigning the task or set of tasks from Staff A to the candidate increases the risk of an adverse event above a threshold. In certain embodiments, one or both of steps 750 and 760 are omitted. If step 750 determines the adverse event risk threshold is exceeded, then this candidate is removed from the candidate pool in step 758. In certain embodiments, a staff member may be allowed exceed an adverse event risk threshold under special conditions, e.g., they are caring for a patient with an infectious disease and the task being reassigned is associated with a patient who is immunocompromised.

If step 760 determines the adverse event threshold is not exceeded, then the system proceeds to step 770 and reassigns the task or set of tasks to the recipient candidate. Step 770 proceeds to step 780 where the system notifies Staff A that their tasks have been reassigned and notifies the candidate of the reassigned task. In certain embodiments, the respective tasks lists of Staff A and the candidate staff member are updated. In certain embodiments, the recipient candidate receives notifications of the tasks, task priority, information identifying the patient and location, and any applicable notes related to the reassigned task.

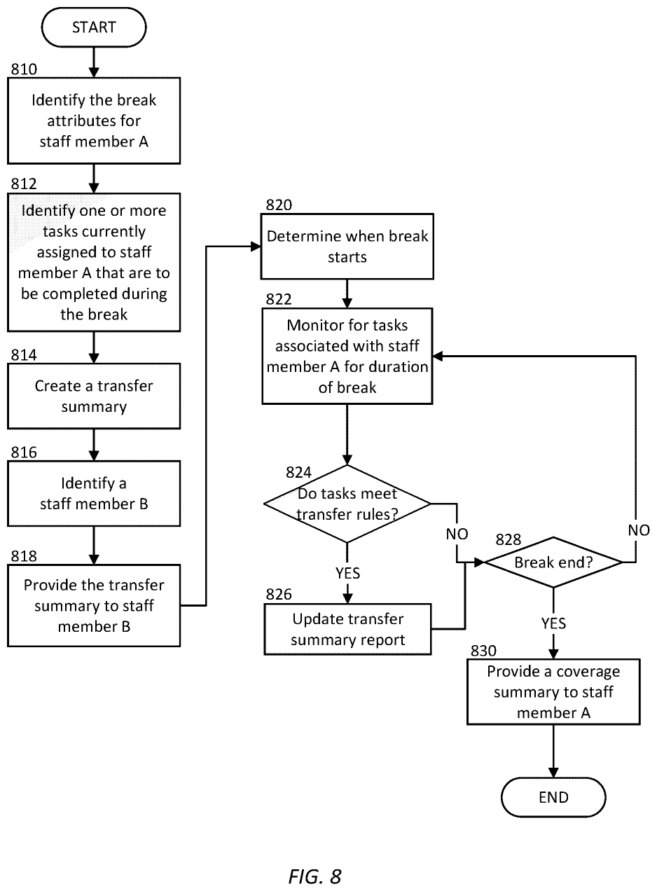

FIG. 8 depicts an exemplary workflow for handing off tasks from one staff member to another staff member, according to certain aspects of the present disclosure. The process starts with step 810 where the Task and Break Management system identifies the break dynamic attributes for staff member A, who is going on break. In certain embodiments, the system pulls one or more break dynamic attributes, e.g., the break start time and break type and break priority and break duration, from the break schedule. In certain embodiments, one or more of these attributes is pulled from a break execution module where the break dynamic attributes may need to be modified in order to execute a break from the break schedule. In certain embodiments, one or more break dynamic attributes are manually entered by a clinician, manager or administrator.

The process proceeds to step 812, and the system identifies one or more tasks that are currently assigned to staff member A that are to be completed during the break. In certain embodiments, the break management systems pulls tasks from staff A's task list that need to be completed by analyzing and comparing one or more task dynamic attributes, e.g., task duration and task expected completion time and task priority, with the break dynamic attributes, e.g., break start time and duration. In certain embodiments, the break type dictates the type of tasks that should be performed during the break by the clinician covering the break, e.g., a break nurse.

In certain embodiments, step 812 identifies possible future ad hoc tasks that have not occurred but would need to be addressed during the break. For example, a task may be created to respond to a medical alarm.

Step 814 creates a break transfer summary report that will be provided to staff member B to inform them of what tasks need to be performed or covered during staff member A's break. In certain embodiments, the break summary report comprises one or more patient dynamic attributes, staff dynamic attributes, task dynamic attributes, and/or break dynamic attributes, e.g., patient identification information, patient location, a task to be completed, a task priority, an expected completion time for a task, and notes from Staff A. In certain embodiments, a break coverage staff member, e.g., a break nurse, covers tasks from multiple staff members taking a break so a break transfer summary report will also identify each staff member who is going on break and their contact information. In certain situations, there are no planned tasks to put on break transfer summary report for a particular patient but there will be a requirement for the coverage staff member to address any ad hoc tasks associated with the patients of the staff on break.

Step 816 identifies a staff member B who will perform the tasks when staff member A goes on break. In certain embodiments, Staff B comprises one person. In other embodiments, Staff B is more than one person and more than one different role. FIG. 7 discusses how the Task and Break Management system identifies staff members to be reassigned tasks.

Step 818 sends the break transfer summary report to staff member B. In some embodiments, the break management system sends this report to various user interface devices and personal communication devices in the hospital, e.g., mobile phone, desktop application, or digital screen outside or inside a patient's room. In certain embodiments, the break coverage staff member receives a notification that they received new information about staff member A going on break and they can see the new tasks when their task list is updated. In certain embodiments, there is a separate task list associated with staff member A that is sent to staff member B's mobile phone. In certain embodiments, staff member B has one task list associated with all the tasks they need to cover. In certain embodiments, staff member B has separate task lists associated with all the staff members they are covering breaks. In certain embodiments, specific categories of tasks, e.g., high priority tasks, are sent as notifications to staff member B as well as an updated task list.

In Step 820, the Task and Break Management system determines when the break starts. In certain embodiments, a break execution module determines the actual break start time when it executes the break schedule for a particular break. For example, staff member A and B each acknowledge the break start time on a user interface and the break execution module tracks the time. In other embodiments, step 820 starts a few minutes before the break starts in an advance notification window as defined in configurable settings in the break execution module.

Once the break starts in step 822, the Task and Break Management system monitors for new or revised tasks associated with the patients that staff member B is covering for staff member A. New or revised tasks can originate from medical devices, e.g. an alarm, other staff members, e.g., a doctor's orders, or other hospital systems, e.g., the pharmacy system, laboratory system, or ADT system.

In step 824, the system determines if any new or revised tasks meet the task transfer rules. Task transfer rules define which new or revised tasks should be covered by the break coverage staff member during the break and which tasks can wait and be addressed by staff A when they come back from break. In certain embodiments, new or revised tasks satisfy break transfer rules and are updated on the break transfer summery report based on the task, task priority, expected completed time of task, and time left on the break. In certain embodiments, the priority of the tasks indicates the urgency with which a task needs to be completed can be the main determining factor for what tasks needs to be completed before the break coverage period ends and therefore added to the break transfer summary report.

In certain embodiments, the task transfer rules incorporate the staff dynamic attributes of the staff members providing the break coverage. For example, a medium or low priority task does not get transferred to the break transfer report if the remaining time of the break is short and the break coverage staff member has a high workload score.

If the break transfer rules are met in step 824, then step 826 updates the break transfer summary report. In certain embodiments updating the break transfer summary report updates the task list for staff B. In certain embodiments previous tasks are replaced with new or revised tasks. In other embodiments, previous tasks are updated with revisions. If break transfers rules are not met in step 824 or if the break summary report is updated in step 826, then the break management system proceeds to step 828 to determine if the break period has ended. If no, then the system loops back to step 822 to continue to monitor for tasks associated with staff member A.

If the break period has ended, then the system branches to step 830 and provides a break coverage summary report to staff member A. The break coverage summary report contains one or more patient dynamic attributes, staff dynamic attributes, and/or task dynamic attributes, e.g., tasks completed, time of completion, person who completed task and contact info, outstanding tasks, origination and received times of outstanding tasks, priority of outstanding tasks, changes in patient's condition, e.g., acuity, and notes from the break coverage person, e.g., conversation with a doctor or a patient request. In certain embodiments, the break summary report is sent to the mobile interface of staff A and updates the task list of Staff A with the outstanding tasks. In certain embodiments, tasks that Staff B still has to complete remain on the task list of staff B and reflected on the break summary report that the task is still outstanding but to be completed by Staff B.

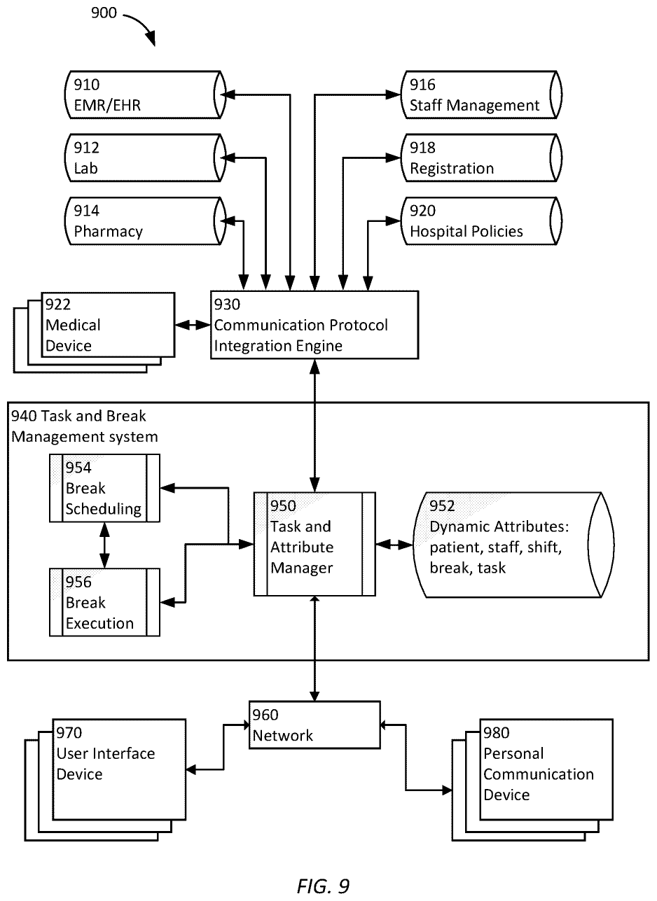

FIG. 9 depicts an exemplary system 900 for managing tasks and breaks, according to certain aspects of the present disclosure. The Task and Break Management system 940 contains data and instructions stored in a memory (not visible in FIG. 9) that, when loaded into a processor (not visible in FIG. 9) of system 940 and executed, cause the processor to implement the functions described herein. In certain embodiments, the system 940 comprises a software application that is hosted on the servers of a Software As A Service (SAAS) provider and accessible by the hospital. In certain embodiments, system 940 comprises an application hosted on a server owned and maintained by a hospital. In certain embodiments, system 940 comprises an application hosted on cloud infrastructure provided by a third party like Amazon Web Services (AWS), Google Cloud, or Microsoft Azure. In certain embodiments, system 940 comprises an application installed directly on personal computers (PCs) located in the hospital. In certain embodiments, system 940 comprises an application developed for mobile devices, e.g. smartphones or tablets, operated within the hospital. In certain embodiments, system 940 comprises a guest application that is hosted on a platform owned by a hospital or a third-party, e.g., medical device manufacturer.

In certain embodiments, system 940 is communicatively coupled to one or more third-party, hospital-based systems, e.g., an electronic health record (EHR) or electronic medical record (EMR) system 910, Lab system 912, Pharmacy system 914, Staff Management system 916, Patient Registration system 918 also referred to as an Admission, Discharge, and Transfer (ADT) system, a Hospital Policies system 920, and connected Medical Devices 922, e.g., an infusion pump or patient health monitor.

In certain embodiments, a Staff Management System 916 comprises one or more of a staff scheduling system, a patient acuity program, a leave and absence management system, a time and attendance system, a human resource information system, a credentialing system, a training and development system, and a performance management system.

In certain embodiments, a Hospital Policies system 920 comprises one or more of a policy management system, a compliance management system, a quality management system, a risk management system, a document control system, a training management system, an audit management system, an incident management system, and a fatigue management system.

In certain embodiments, system 940 communicates bidirectionally with these systems through a Communication Protocol integration engine 930 ensuring that data from the hospital-based systems is translated appropriately for system 940 and vice versa. Communication is accomplished using any defined communication protocol, e.g., Health Level 7 (HL7). In certain embodiments, one or more communication links are unidirectional.

System 900 is an exemplary representation of how system 940 is integrated into a system but other system architectures that perform the same functions are considered equivalent. In the embodiment of FIG. 9, the Task and Attribute Manager 950 receives tasks and dynamic attributes from the hospital systems and medical devices and sends this information to other modules and databases within the application. In certain embodiments, the Task and Attribute Manager 950 can create task lists, communicate attributes and task information to the Break Scheduling 954 and Break Execution 956 modules, and communicate break schedules, break execution notifications, alerts and alarm messages, and task lists through a communication protocol via a Network 960 to either a Personal Communication Device 980, e.g., a mobile phone, or a User Interface Device 970, e.g., a central nurse workstation.

The Break Scheduling module 954 creates and continuously revises break schedules using the break, shift, patient, and staff dynamic attributes that the Task and Attribute Manager 950 pulls from the Dynamic Attribute Database 952 and internal break scheduling rules. The Break Execution module 956 executes break schedules when schedules need to be executed by retrieving the break schedule and the attributes associated with the schedule from the Break Scheduling module 954 and communicating with the Task and Attribute Manager 950 to determine what task to reassign from various staff members to execute the breaks. The Break Execution module 956 has internal rules to determine how to execute breaks. In certain embodiments, system 940 allows hospital clinicians, managers, and administrators to create break scheduling, break execution, and task reassigning rules via a user interface 970.

The Dynamic Attribute Database 952 is constantly updating with new attribute information from systems 910, 912, 914, 916, 918, 922, 922. The Task and Attribute Manager 950 pulls the most recent dynamic attributes from one or more of these systems to send to the Break Scheduling module 954 to create breaks and to the Break Execution module 956 to execute breaks by determining how to reassign tasks, if necessary.

The system 940 processes inputs through User Interface Devices 970 and Personal Communication Devices 980. In certain embodiments, a staff member requests a break at a User Interface Device 970 or Personal Communication Device 980 and the Task and Attribute Manager 950 sends the request to the Break Execution module 956 to execute and to the Break Scheduling module 954 to revise the break schedule. In certain embodiments, the Task and Attribute Manager 950 sends a break request from a staff member to another User Interface Device 970 interface to get a supervisor's approval before sending to the Break Execution module 956 to execute.

FIG. 10 depicts a flowchart of an exemplary method of managing breaks, according to certain aspects of the present disclosure. It is the intent of the disclosed methodology to provide breaks for more than one staff member, e.g., 2 or more nurses, to take a concurrent break in order to ensure that all staff members get breaks and potentially improve the quality and benefits of a break by enabling multiple staff members to take breaks together. This concept covers a single staff member taking a break by themselves, with their patients being covered by one or more other staff members, as well as the concept of a Break Nurse (BN), whose role is primarily to provide coverage of patients while other staff take a break. In certain embodiments, the BN is a special category of a staff nurse who does not have any assigned patients themselves. While the presence of a BN ensures that any one clinician can take a break, use of the disclosed method enables more than one clinician to go on break at the same time if there is sufficient bandwidth available from other clinicians to cover the patients.

The process starts with step 1010 wherein a group of one or more first clinicians (FCs) are identified to take a concurrent break. Step 1020 determines the total pool of patients that will need to be covered if all of the FCs identified in step 1010 are on break. In certain embodiments, the determination of the total pool of patients comprises determining one or more patient attributes, e.g., patient location, patient acuity, and patient care tasks and associated windows of time for completing the respective tasks.

Step 1030 selects one or more second clinicians (SCs) as candidates to cover one or more of the total pool of patients identified in step 1020. The group of SCs is excusive of the FCs, although the SCs will generally have similar roles and skill sets to the FCs in order to be able to provide coverage of their patients. In certain embodiments, one or more of the SCs have a lower role and are assigned only to patients requiring less care, e.g., patients having a lower acuity. In certain embodiments, one or more of the SCs have a higher role and are assigned patients requiring more care, e.g., patients having a higher acuity.

Each of the SCs has one or more break coverage attributes of a group that consists of:
a maximum patient-staff ratio (MPSR);
a maximum patient acuity;
a maximum number of patients having an acuity that is less than or equal to a threshold acuity;
a maximum aggregate patient acuity;
a maximum distance between assigned patients;
a break priority
a break status
a maximum number of tasks to be completed during the concurrent break; and
a Do Not Disturb (DND) status.

In certain embodiments, a care facility has a policy that sets an MPSR for all clinicians in a certain unit, e.g., nurses on a MED-SURG unit of hospital X have an MPSR of 5. In certain embodiments, a law or regulation establishes a MPSR for all care facilities within the jurisdiction of the entity issuing the law or regulation. In certain embodiments, the MPSR is associated with the role of the clinician, e.g., a junior nurse on a unit has an MPSR of 3 while a primary nurse has an MPSR of 5. In certain embodiments, an SC has an MPSR that considers all the patients being covered by the SC but a lower limit on the number of patients that the SC may be assigned to cover. For example, a lead nurse may have an MPSR of 5 to include all patients being care for by the SC, including patients for which the SC is the primary care giver, and a second lower MPSR for the maximum number of patients that the SC can temporarily cover for another clinician. In certain embodiments, the second MSPR is set by the respective SC. In certain embodiments, only clinicians having an ability to accept a temporary re-assignment of a patient to cover are selected in step 1030.

In certain embodiments, the group of selected SCs includes one or more Break Nurses (BNs), whose role is to cover patients so that the regular staff clinicians, who are assigned as primary care provides to patients, can take a break. In certain embodiments, the MPSR of a BN is equal to the highest MPSR allowed by law, regulation, and facility policy. In certain embodiments, patients are first re-assigned to the BN up to the MPSR of the BN before any patient is re-assigned to another SC, so as to minimize the extra work asked of clinicians who have their own assigned patients. In certain embodiments, patients are re-assigned to the BN in order of the patient acuity, e.g., the patients with the highest acuity are assigned to the BN first. In certain embodiments, patients are re-assigned to the BN in proximate groups, e.g., re-assigned patients are within a defined maximum distance of each other even if the patients are being re-assigned from different FCs.

Step 1032 temporarily re-assigns the total pool of patients to the SCs selected in step 1030. In certain embodiments, patients are incrementally re-assigned based in part on their respective acuity, e.g., the highest acuity patients are assigned first. In certain embodiments, re-assignment of a patient to a SC must comply with the limits of their respective break coverage attributes, e.g., a patient cannot be assigned to an SC when the patient's acuity is greater than the maximum patient acuity of the SC's break coverage attributes. In certain embodiments, a patient is tentatively assigned to an SC even though one or more break coverage attributes are violated and the tentative assignment is submitted to a supervisory for approval to override the violation.

Once all possible re-assignments of patients has been completed in step 1032, step 1034 determines whether any patients have not been re-assigned, e.g., identifies patients as belonging to a “remaining pool of patients” by removing the re-assigned patients from the total pool of patients. If all of the patients of the FCs can be covered by the selected SCs, the number of patients in the remaining pool is zero.

Step 1036 branches to step 1060 if the number of patients in the remaining pool is zero. Step 1060 notifies the selected FCs to take the concurrent breaks and the selected SCs of the patients that each of them is to cover. In certain embodiments, the proposed FCs and re-assignments to SCs is presented to a supervisor for approval prior to execution of step 1060. If there are any patients in the remaining pool, the process branches to step 1040 to consider the impact of a Charge Nurse (CN) or other senior clinician that may be able to cover one or more of the patients in the total pool.

In certain embodiments, steps 1030-1036 are performed multiple times in parallel to consider the impact of selecting different candidate groups of SCs. In certain embodiments, step 1036 comprises comparisons of the multiple different groups of SCs, e.g., which groups of SCs provide all necessary coverage, and selection of one of the groups of SCs for implementation. In certain embodiments, each candidate FC is assigned a break priority and the comparison comprises an evaluation of the FC break priorities, e.g., a group of FCs that includes the FC having the highest break priority is preferred in step 1036 over a group of FCs having lower priorities. In certain embodiments, the parallel evaluation includes steps 1040-1050 to consider the impact of an available CN on each of the candidate groups of FCs.

Step 1040 determines whether a CN is available. While this example process is presented in the context of a nursing unit having a CN overseeing the unit, wherein the CN is a senior clinician with the skills and experience to perform any task within the unit, this same concept is generally applicable to any situation having a senior staff member who is not regularly assigned the same tasks as their staff but has the ability to cover a responsibility of one of their staff. In certain embodiments, this determination comprises an evaluation of the CN's break coverage attributes with respect to one of more of the patients to be covered. In certain embodiments, this evaluation is performed with respect to a patient in the remaining pool. In certain embodiments, this evaluation is performed with respect to one or more of the patients in the original total pool of patients, regardless of whether that patient has been tentatively re-assigned in steps 1010-1036. In certain embodiments, the process branches to step 1042 if any patient in the total pool can be covered by the CN.

A CN has their own set of break coverage attributes. In certain attributes, the CN sets one or more of the values of their break coverage attributes. For example, a CN may decide that they can accept only 1 patient to cover in addition to their own workload, which does not otherwise include direct patient care, and sets their MPSR to 1. In another example, the CN may decide that they can accept only patients within a certain distance of their office and sets the maximum distance of an assigned patient relative to the office location. In certain embodiments, the break coverage attributes of the CN are more limiting than the break coverage attributes of the other SCs. In certain embodiments, the CN has a set of break coverage attributes that prevents them from covering any of the total pool of patients.

The CN may have a set of previously assigned tasks or responsibilities that cannot be accomplished while the CN is covering a patient. Optional step 1044 re-assigns one or more tasks previously assigned to the CN to another clinician, e.g., one of the SCs. In certain embodiments, the SC receiving the re-assigned task is not assigned to cover a patient, e.g., is additional to the SCs selected in step 1030.

In certain embodiments, the CN is assigned an assistant who can execute one or more previously assigned tasks and/or provide a portion of the coverage of a patient. In certain embodiments, the break coverage attributes of the CN have a second maximum number of tasks to be completed during the concurrent break if an assistant is assigned to the CN.

Step 1046 determines a new remaining pool of patients by removing patients assigned to the CN from the total pool in addition to removing patients assigned to the SCs.

Step determines whether there are any patients in the remaining pool, i.e., is there a patient who will not be covered if the proposed concurrent break is implemented? If all patients will be covered, i.e., zero in the remaining pool, then process branches to step 1062 and the FCs, SCs, and CN are appropriately notified. If a patient will not be covered by the proposed re-assignments, the process branches back to step 1052 to select a new set of FCs.

In this example process, step 1042 selects a patient from the total pool for re-assignment to the CN without regard to whether this patient was tentatively re-assigned in prior steps of the execution of this process. In certain embodiments, the selection of a patient for re-assignment to the CN minimizes the impact on the CN. In certain embodiments, the number of patients re-assigned to the CN is equal to the number of patients in the remaining pool in step 1036, i.e., just enough to fill the gap between the total pool of patients and the number of patients that can be covered by the SCs.

If the CN is unable to accept any of the total pool of patients in step 1040, the process branches to step 1052 that selects a different set of FCs for evaluation for a concurrent break. If the CN can accept at least one patient from the total pool of patients, the process branches to step 1042 that assigns one or more patients from the total pool to the CN.

In certain embodiments, step 1042 attempts to assign the patients in the remaining pool to the CN then execute steps 1046 and 1050. If the CN can accept these patients, the process reaches step 1062 and instructs the FCs of the selected group to take the concurrent break while informing the SCs and CN of the patients that they are to respectively cover.

In certain embodiments, step 1042 assigns one or more patients from the total pool to the CN then re-assigns the other patients of the total pool to the SCs. In certain embodiments, a particular set of re-assignments is identified and then steps 1046-1050 are executed to determine whether this set of re-assignments meets all criteria, wherein the process branches to step 1062. In certain embodiments, multiple sets of re-assignments, e.g., different patients are re-assigned to the CN or SCs, are evaluated in parallel and steps 1046-1050 compare the multiple sets, e.g., which set of re-assignments meet all break coverage attributes of all SCs and the CN, and one of the evaluated sets is selected for execution in step 1062.

If step 1050 determines that the set of re-assignments of patients to the CN and SCs does not cover all patients, the process branches to step 1052 to select a new set of FCs and repeat the processes starting at step 1020. In certain embodiments, step 1050 notifies a supervisor (not shown in FIG. 10) if it is found that there is no combination of SCs and CN that enables the selected FCs to take a concurrent break. In certain embodiments, the supervisor is the CN.

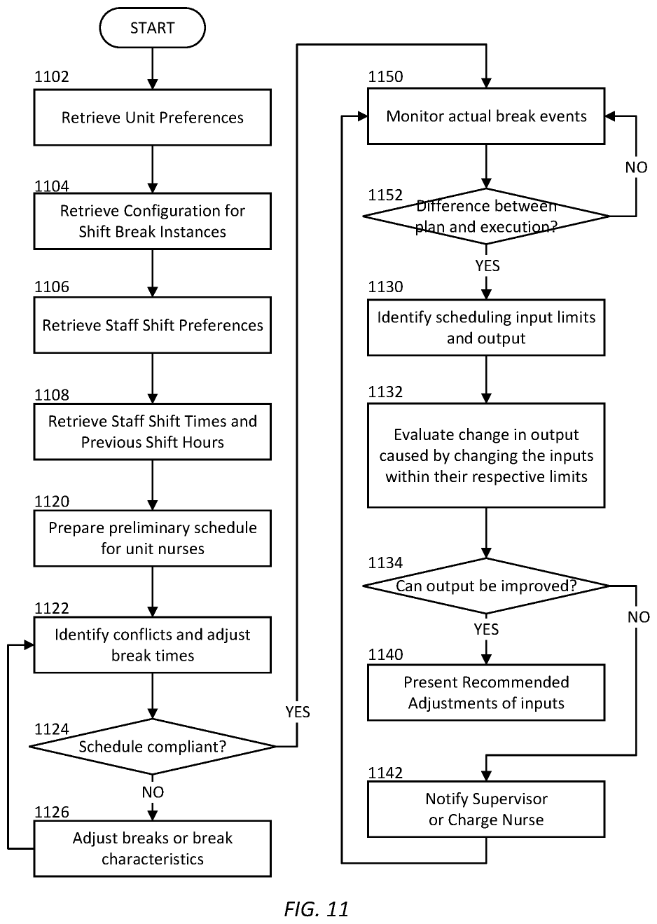

FIG. 11 depicts a flowchart of an exemplary method of optimizing selected characteristics of the schedule, according to certain aspects of the present disclosure. It is the intent of the disclosed methodology to provide breaks for all staff members in a unit while complying with applicable state codes and facilities policies as well as honoring, to the extent possible, preferences determined by the individual staff members. This method applies to nurse break systems that implement concurrent breaks as described above, with single breaks models that use break nurses or use break buddies (i.e. the break coverage nurse covers both her own patients and the patients of the nurse she is providing the break coverage for).

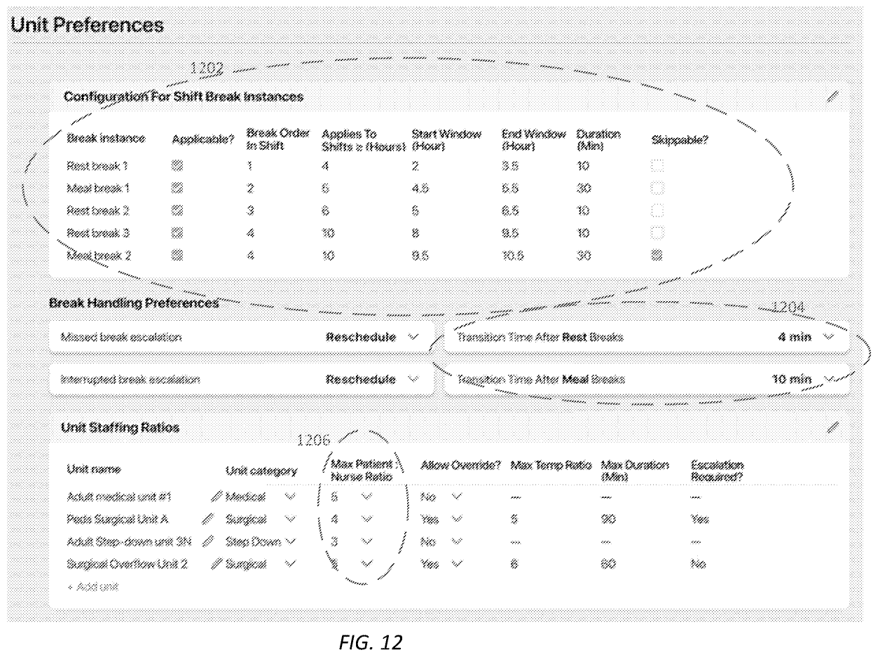

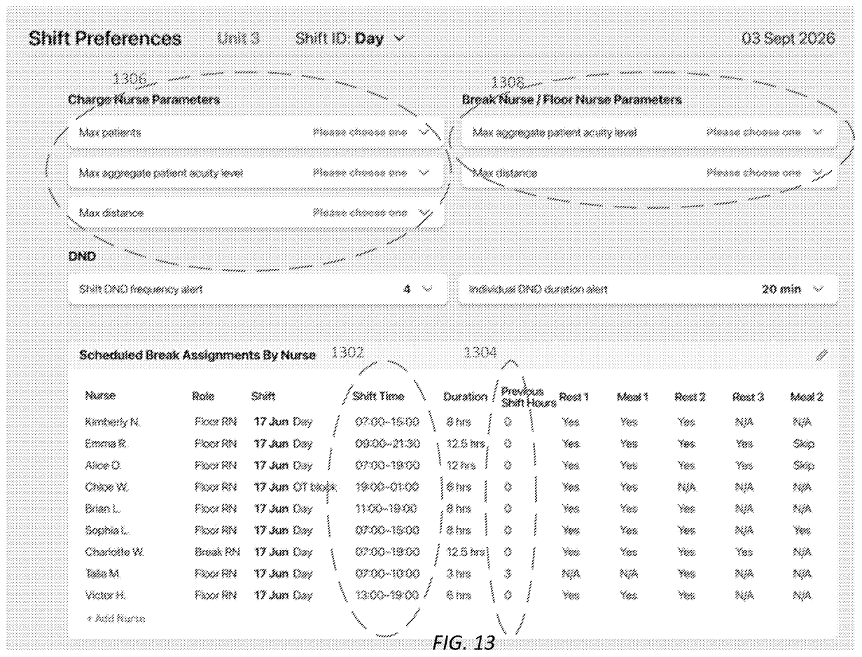

Steps 1102-1108 retrieve information regarding state codes (see FIG. 12, 1202), unit preferences (see FIG. 12, 1204), staff shift preferences (see FIG. 13, 1306, 1308), and staff shift assignments (see FIGS. 13, 1302 and 1304).

Step 1120 prepares a first draft schedule, e.g., by selecting break times individually for each nurse without regard for staff coverage or conflict between nurse schedules.

Steps 1122-1126 evaluate the draft schedule for ability of each nurse to take their breaks, adequacy of coverage, and iteratively adjust one or more of the times, durations, and types of breaks within the constraints of unit and staff shift preferences. In certain embodiments, there is a limit to the number of iteration loops (from step 1126 back to 1122) that are allowed before the process branches to step 1142 (this path not shown for clarity) and notifies the supervisor or charge nurse that a schedule that meets all requirements cannot be found.

Steps 1150-1152 monitor execution of the break schedule. If there is a variance, for example a nurse is late coming back from a break or deferred a break because of an urgent task, the process branches to step 1130 that identifies the inputs, e.g., staff preference for maximum patient acuity or charge nurse preference for maximum number of patients to be assigned, and the output, e.g., number of concurrent breaks during shift.

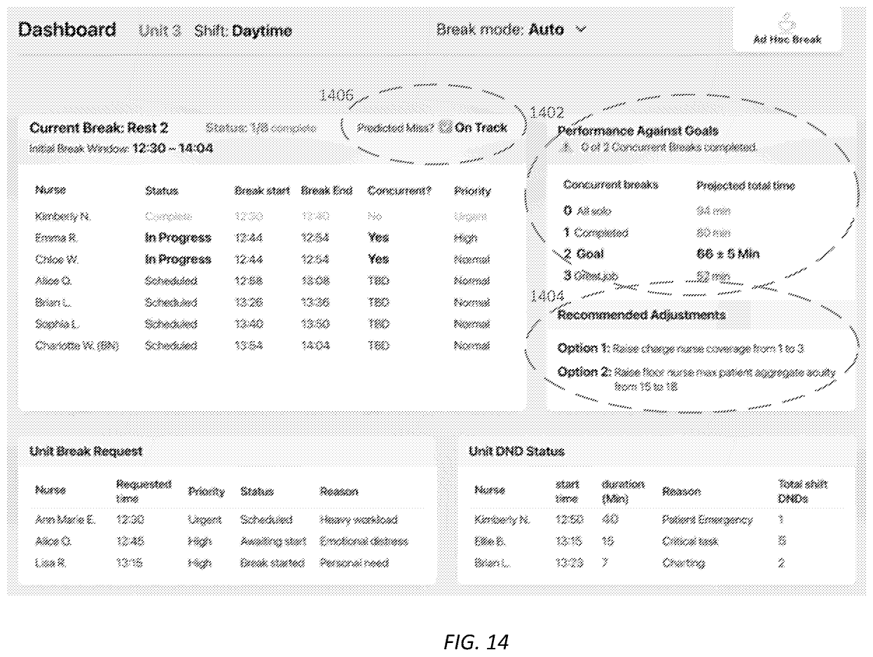

Steps 1132-1140 evaluate the effects of adjusting the inputs from the values used for the current schedule and then re-run the schedule evaluation to determine if the output can be improved by one or more changes in the inputs. If it is possible to improve the output, the recommendations are presented to the user via a user interface (see FIG. 14, 1404). If it is not possible to remain in compliance with all constraints, e.g., code and unit preferences, then the process branches to step 1142 and notifies the supervisor, e.g., charge nurse. In either case, the process then returns to step 1150 and monitors for further changes.

FIG. 12 depicts an exemplary user interface screen including unit preferences, according to certain aspects of the present disclosure.

Area 1202 depicts an exemplary display showing the user entry of state code requirements, specific legal requirements for number of breaks based on duration of shift, timing of breaks, duration of breaks, and whether staff members are allowed to skip or decline certain break instances (e.g. meal break 2). These may be preconfigured to meet state regulations or configured by the facility to meet hospital policy or state regulations. In certain embodiments, the parameters of the state regulations are manually entered by the customer, e.g., the hospital administration. In certain embodiments, the state regulations are provided by the software as delivered to a particular customer, e.g., customized to local laws and regulations by a third-party as a service to the hospital. In certain embodiments, additional columns may be added to this table such as when break must start by or end by to ensure breaks meet the state specific regulatory requirements for when a break must start or end (e.g. some states require the first meal break must start by the fifth hour of a shift).

Area 1204 provides a means for a user, e.g., and administrator, to enter values for transition times from break to work for meal breaks or rest break, e.g., 10 minutes transition between the end of one meal break and the start of a work period thereby recognizing the time it takes to catch up on changes and activity that occurred during the break.

Area 1206 displays the current values and allows user entry of maximum patient staff ratios for each individual unit and type of unit to meet state regulations or hospital policies. For example, a medical unit might allow a nurse to have a maximum of 5 patients while an ICU floor will allow only 1 or 2 patients per nurse. The Unit Staffing Ratios table also allows nurse managers to temporarily deviate from state or hospital defined ratios in the event of an emergency, e.g., short staff, presence of codes on the unit, etc. If the hospital allows a temporary override of a maximum patient staff ratio, then nurse managers can select the max temporary patient staff ratio, the duration for this override, and if this override first requires an escalation to the charge nurse for approval.

FIG. 13 depicts an exemplary user interface screen including staff preferences, according to certain aspects of the present disclosure.

Area 1302 shows each nurse's assigned shift. In certain embodiments, the shift hours are retrieved from another hospital system, e.g., a time and attendance system. In certain embodiments, the software calculates the duration of hours worked in the shift or consecutive shifts and identifies whether the nurse is working overtime.

Area 1304 displays hours worked by each nurse immediately prior to the shift being scheduled, e.g., some nurses might be called in early to work a portion of the prior shift to cover for a sick nurse who went home early. Based on the total number of hours worked, the software will display how many breaks and which breaks a nurse deserves based on calculations from Section 1202.

Area 1306 enables the charge nurse to select their preferences and, once entered, displays the selected values. These preferences provide the break algorithm with information on how many patients the charge nurse is willing to cover during a break, what is the maximum aggregate acuity they are willing to take, and the max distance they are willing to cover. In other embodiments, the charge nurse can decide what level of workload they are willing to take based on active tasks in the EHR or the historical amount of tasks performed on the patient. Area 1308 enables a break nurse and/or a staff nurse to select their preferences. In certain embodiments, these selections are entered by the charge nurse or nursing manager. In certain embodiments, there are additional and/or alternate preferences, e.g., maximum workload, or the maximum number of tasks (e.g., tasks from the EHR) that the nurse is allowed to cover during a break coverage period.

FIG. 14 depicts an exemplary user interface screen including performance and recommendations, according to certain aspects of the present disclosure.

Area 1402 displays the current goal performance report and projected performance based on current schedule plan. In certain embodiments, these parameters are scheduling outputs. In this example, the goal is to get two concurrent breaks during the “rest 2” break instance. Rest 2 will take 94 minutes to complete with 7 nurses each needing a 10-minute break and there is a scheduled 4-minute transition between each break if all breaks are single breaks. The Performance Against Goals Table 1402 shows for each concurrent break executed will reduce the total break time by 14 minutes. In other embodiments, the software can take a break window perspective (e.g. a two-hour window from 9:00 AM to 11:00 AM) instead of a break instance specific approach. Since nurses start their day at different hours, some nurses may have different break instances during the same break window (e.g. one nurse may have their rest break 1 while another nurse may have their meal break 1 during the 9:00 to 11:00 AM window. In this embodiment, the concurrent break goals apply to the break window instead of a specific break instance.

Area 1404 displays exemplary predictive analytic recommendations developed by the software to help guide the charge nurse on how to improve concurrent break performance. The process of FIG. 10 shows how maximum patient staff ratios and other break coverage attributes, e.g., acuity and distance, need to be met in order to assign concurrent break coverage to charge nurses, break nurses and floor nurse. For the recommended adjustments, the software will look at the shift preferences and unit preferences and determine how changing these parameters can lead to better break performance outcomes across a break instance or a break window (e.g. 9:00 to 11:00 AM). For example, the charge nurse can achieve more concurrent breaks for Rest 2 if she raises her charge nurse coverage to cover 3 patients per break instead of 1 or if they raise the maximum aggregate acuity from 15 to 18. In certain embodiments that includes workloads, e.g., the number of tasks from the EHR that need to be complete during the break coverage period, the software makes one or more recommendations to defer all low and medium priority tasks until after the break coverage period so nurses can take on more patients to execute additional concurrent breaks.

Area 1406 provides a summary prediction by software as to whether all nurses or specific nurse will be able to take their required breaks during a break instance, e.g. Rest 2. A unit may not be able to maintain the order and timeliness of executing a break for a number of reasons: nurses come back from breaks late, nurses defer breaks by allowed limits, e.g., 3 minutes, a code is called in the unit that required the charge nurse to temporarily pause the break algorithm and the break executions. In certain embodiments the software will predict for each individual nurse whether they are “at risk” to miss their break or have a late break based on how the charge nurse enters the information in Section 1202. For example, the software may predict a nurse is at risk to not get her break by the start time required by their state regulations (e.g. a nurse must take their first meal break by the fifth hour of the shift). In certain embodiments the software may determine the nurse is at risk of getting a late break based on how many nurses have a higher priority break in front of the nurse and how much time is left before this nurse needs to take their break before required by hospital or state regulations.

In certain embodiments, the “predictive miss” feature determines if nurse will miss their breaks by the scheduled end time of the break window. In certain embodiments, the “predicted miss” feature indicates “on track” or “not on track” and, if not on track, makes more aggressive recommendations for the particular break instance or a specific break window to improve the number of concurrent breaks performed.

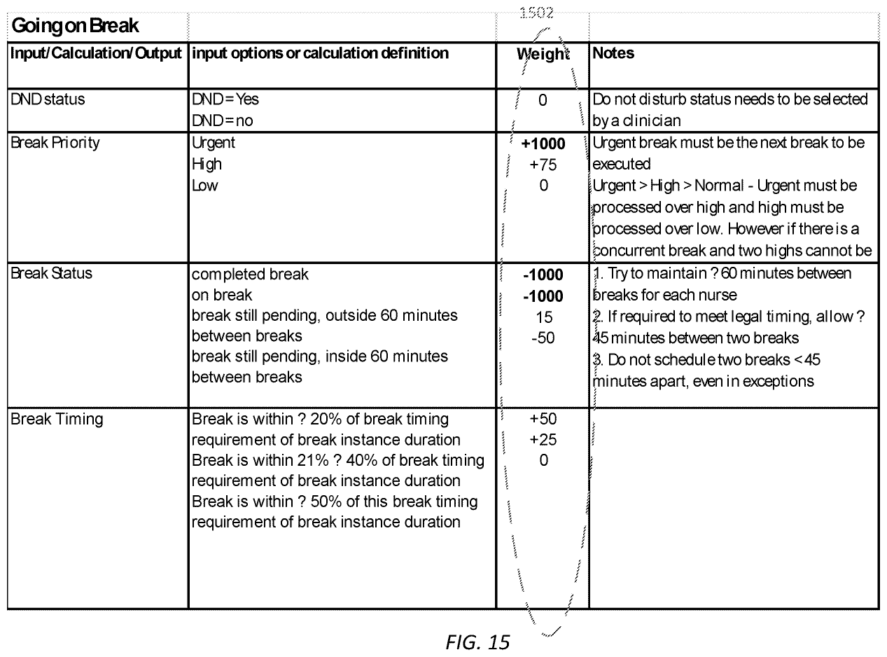

FIG. 15 depicts a table containing exemplary weightings used to determine who goes on break, according to certain aspects of the present disclosure. In certain embodiments, the table inputs go into calculating who should be the next person to go on break. In certain embodiments, other parameters (not shown in FIG. 15) are used to determine who should go on break next, e.g., a nurse manager requests Nurse A to be the next nurse to go on break.

Area 1502 displays exemplary weightings to be used in an algorithm to rank nurses as to who should take the next break. Positive numbers indicate a need, with larger numbers related to greater need, and negative numbers minimize the need for a break. The highest cumulative weight indicates who should go on break next. In certain embodiments, an applied weight of −1000 indicates that the nurse is not eligible to take a break in the given break instance.

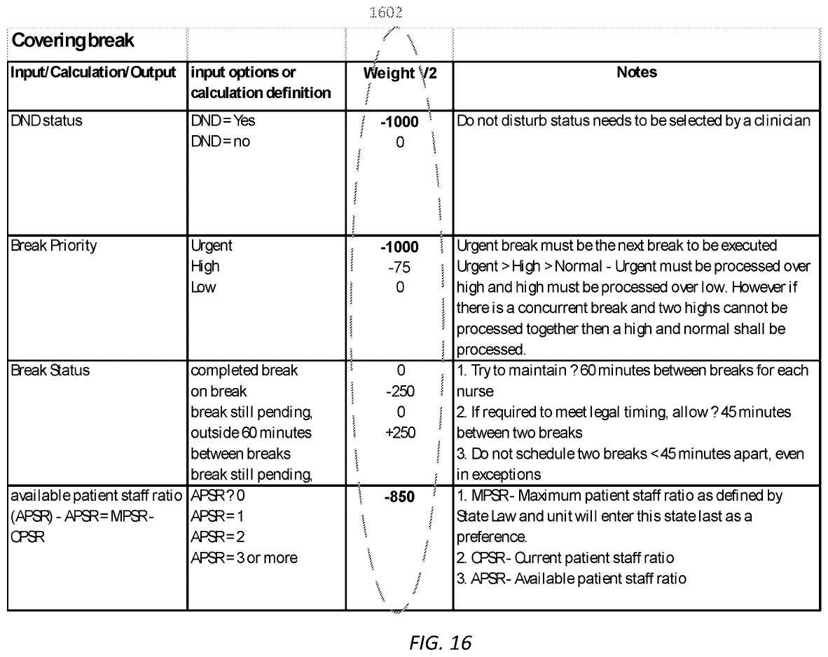

FIG. 16 is a table containing exemplary weightings used to determine who covers for a break, according to certain aspects of the present disclosure. In certain embodiments, the table shows inputs that go into calculating who should be the nurse(s) to provide break coverage for the nurse going on break. In certain embodiments, other parameters (not shown in FIG. 16) are used to determine who should cover the next break, e.g., an anticipate nurse workload during the break coverage.

Area 1602 depicts exemplary weightings used in an algorithm to rank nurses as to who should cover a particular break, with larger numbers related to a greater capacity to provide coverage, and negative numbers indicate an inability to provide coverage.

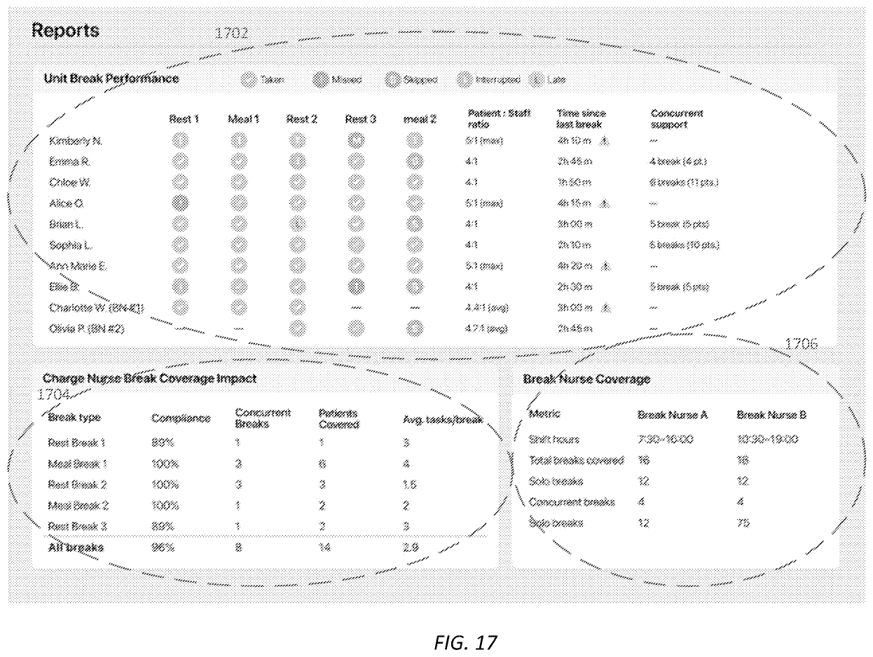

FIG. 17 depicts an exemplary user interface screen displaying unit and key individual performance, according to certain aspects of the present disclosure.

Area 1702 presents information related to whether nurses are getting their scheduled breaks. In certain embodiments, the interface tracks individual nurses and their breaks and whether the breaks were taken, skipped, or missed. In certain embodiments, the time-since-last-break is tabulated and values above a predetermined threshold are marked. In certain embodiments, this table shows how much support each floor nurse gave for executing concurrent breaks.

Area 1704 reports the charge-nurse burden in supporting each break and compliance rate achieved. In certain embodiments, the compliance parameter is a scheduling output. In certain embodiments, this table shows how much support each charge nurse gave for executing concurrent breaks including the number of patients covered and tasks performed during the concurrent break coverage, which allows the charge nurse to see how their contributions allowed for more concurrent breaks,

Area 1706 reports the break nurses' support of breaks, thereby providing a measure of the effectiveness of a break nurse on the particular shift.

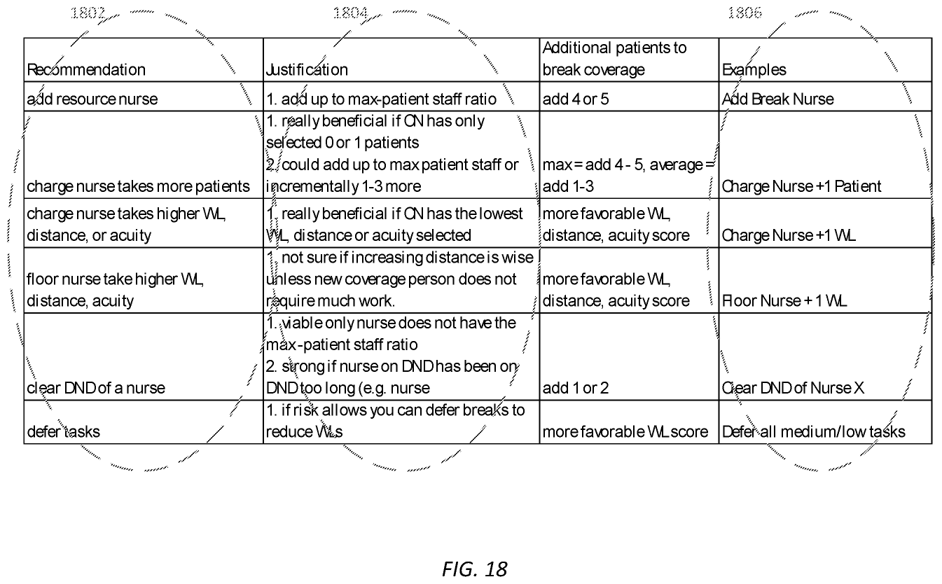

FIG. 18 depicts exemplary recommendations, according to certain aspects of the present disclosure.

Area 1802 lists exemplary recommendations that the software can present to increase the number of concurrent breaks during a particular break instance or for all breaks on a shift. In certain embodiments, these recommendations improve single-break coverage in either the break-buddy model, e.g., wherein one nurse covers both her patients and the patients of the “buddy” nurse going on break during the break coverage, or a break-nurse model, e.g., wherein all single breaks are covered by break nurse.

In certain embodiments, the software makes recommendations, e.g., recommends that the charge nurse cover patients for a nurse taking a break or recommends that the unit brings in a temporary float nurse to assist.

Area 1804 depicts an informational rationale and considerations for presenting this recommendation. In certain embodiments, this information is entered by the administrator who is configuring the software as to what recommendations can be made.

Area 1806 depicts informational examples of specific recommendations, e.g., what can be accomplished based on the staff capabilities and type of patients in the unit.

### SUMMARY

The disclosed systems and method automatically analyze schedules and patient assignment records and temporarily re-assign patients to enable one or more clinicians to take a concurrent break. In certain embodiments, the system determines whether a senior staff member, e.g., a Charge Nurse, can make up the difference if the available staff cannot provide complete coverage of patients as required for a group of clinicians to take a concurrent break.

As used in the description of the disclosure and the appended claims, the singular forms “a,” “an” and “the” and the like are intended to include to be interpreted as equivalent to the phrase “at least one” and comprise the plural forms as well, unless the context clearly indicates otherwise. Reference to an element in the singular is not intended to mean “one and only one” unless specifically so stated.

As used in the description of the disclosure and the appended claims, pronouns in the masculine (e.g., his) include the feminine and neuter gender (e.g., her and its) equivalent and vice versa.

As used herein, “and/or” refers to and encompasses any and all possible combinations of one or more of the associated listed items as well as singular usage of each item.

As used herein, the terms “aspect” and “embodiment” are used to identify examples as to how the disclosure may be utilized and do not necessarily identify features or components that essential to the subject technology or that apply to all configurations of the subject disclosure. A disclosure relating to an aspect may apply to all configurations, or one or more configurations.

As used herein, the term “exemplary” means “serving as an example or illustration.” Any aspect or design described herein as “exemplary” is not a limiting embodiment nor to be construed as preferred or advantageous over other designs.

As used herein, the terms “include,” “contain,” “has,” “have,” and the like are intended to be inclusive in a manner similar to the term “comprise” as interpreted when employed as a transitional word in a claim. Claim language or other language in the disclosure reciting “at least one of” a set and/or “one or more” of a set indicates that one member of the set or multiple members of the set (in any combination) satisfy the claim. For example, claim language reciting “at least one of A and B” or “at least one of A or B” means A, B, or A and B. In another example, claim language reciting “at least one of A, B, and C” or “at least one of A, B, or C” means A, B, C, or A and B, or A and C, or B and C, or A and B and C. The language “at least one of” a set and/or “one or more” of a set does not limit the set to the items listed in the set. For example, claim language reciting “at least one of A and B” or “at least one of A or B” can mean A, B, or A and B, and can additionally include items not listed in the set of A and B.

Headings and subheadings, if any, are used for convenience only and do not limit the invention.

### Illustrative Embodiments

A1. A method comprising steps of determining that a first staff member needs a break having attributes of a start time and a duration; automatically identifying a task that is assigned to the first staff member and conflicts with the break dynamic attributes; and notifying the first staff member, if no conflicting task is identified, to take the break at the start time.

A2. The method of A1, further comprising the steps of determining, if a conflicting task is identified, whether the break can be delayed long enough to complete the identified task; adjusting, if it is determined that the break can be delayed, the start time to allow completion of the identified task; and notifying the first staff member, if the start time is adjusted, to complete the identified task then take the break.

A3. The method of A2, further comprising steps of automatically selecting, if it is determined that the break cannot be delayed, a second staff member that can perform the identified task based in part on a staff dynamic attribute of the second staff member; re-assigning the identified task to the second staff member; and notifying the first staff member, if the task is re-assigned, to take the break at the start time.

A4. The method of A3, wherein the break cannot be delayed if any of the following occur: an input is received from a supervisor that the first staff member must take the break at the start time; a continuous work time of the first staff member from an end of a last break to the start time exceeds a first threshold; and a current fatigue level of the first staff member exceeds a second threshold.

A5. The method of A3, wherein the staff dynamic attribute of the second staff member comprises one or more of a group consisting of a workload; a fatigue score; and the step of selecting the second staff member comprises evaluating whether re-assigning the identified task to the second staff member will increase one of more of the workload and the fatigue score of the second staff member above a respective threshold.

A6. The method of A1, wherein the step of determining that the first staff member needs the break comprises accepting a schedule for the first staff member that comprises the break, the start time, and a duration of the break.

A7. The method of A1, wherein the step of identifying a task comprises accepting a list of one or more uncompleted tasks assigned to the first staff member; identifying a task on the list that has a task duration and a time window; automatically comparing the task duration and the time window of the identified task to the start time and the duration of the break; and determining that the identified task conflicts with the break if the identified task cannot be completed before the start time and the identified task cannot be completed within the time window if the identified task is started after the break is completed.

A8. The method of A7, wherein the identified task cannot be completed before the start time when the identified task has not been started and the identified task has a total task duration that is greater than a time difference between a current time and the start time.

A9. The method of A7, wherein the identified task cannot be completed before the start time when the identified task has been started and the identified task has a remaining task duration that is greater than a time difference between a current time and the start time.

A10. The method of A1, wherein the step of determining that the first staff member needs the break comprises receiving a request for a break at a proposed start time with a proposed duration.

A11. The method of A10, wherein the request comprises changing the start time to the proposed start time.

A12. The method of A11, wherein the request comprises changing the duration to the proposed duration.

A13. The method of A12, wherein the request to change the duration comprises a request to end a current work shift at the proposed start time.

A14. The method of A10, wherein the request is received from the first staff member.

A15. The method of A1, wherein the step of determining that a first staff member needs a break is based in part on a staff dynamic attribute of the first staff member.

A16. The method of A3, further comprising the step of receiving, after the start time, a new task that is assigned to the first staff member; re-assigning the new task to the second staff member.

A17. The method of A2, further comprising the step of changing, if it is determined that the break cannot be delayed, an attribute of the break that is one or more of a group of attributes comprising the start time, the duration, and a type of break.

A18. Thew method of A1, wherein: a task conflicts with the break if the task cannot be delayed until the break is completed.

B19. A non-volatile machine-readable memory comprising instructions that, when loaded into a processor and executed, cause the processor to perform the method of A1.

C20. A system, comprising a processor and a non-volatile machine-readable memory comprising instructions that, when loaded into the processor and executed, cause the processor to perform the method of A1.

D1. A method of enabling a staff member to change a break dynamic attribute, comprising steps of requesting a modification of a schedule; determining whether the modified schedule complies with rules and constraints; and implementing, if the modified schedule complies with the rules and constraints, the modified schedule.

D2. The method of D1, further comprising determining whether a task re-assignment would resolve a conflict between the modified schedule and the rules and constraints.

D3. The method of D1, further comprising determining whether a modification of a break dynamic attribute would resolve a conflict between the modified schedule and the rules and constraints.

D3. The method of D1, wherein the modification of the schedule comprises a change in an attribute of a future break.

D4. The method of D3, wherein the break dynamic attribute comprises one or more of a break start time, a break duration, a break type, a break priority, and a location of the break.

E1. A method of re-assigning a task currently assigned to a first staff member, comprising steps of selecting a task to be re-assigned; identifying a pool candidates capable of performing the selected task based in part on one or staff dynamic attributes; determining whether re-assigning the task to a candidate selected from the pool of candidates will raise a dynamic attribute of the selected candidate above a first threshold; and re-assigning the task, if it is determined that re-assigning the task to the selected candidate will not raise the dynamic attribute above the first threshold, to the selected candidate.

E2. The method of E1, further comprising determining whether re-assigning the task to a candidate selected from the pool of candidates will raise a risk of an adverse event above a second threshold; and re-assigning the task, if it is determined that re-assigning the task to the selected candidate will not raise the risk of an adverse event above the second threshold, to the selected candidate.

F1. A method of handing off a task while a first staff member takes a break, comprising steps of identifying a task to be performed while the first staff member is on the break, the identification based in part on a task dynamic attribute of the task; creating a task transfer report comprising information about the identified task; identifying a second staff member who is capable of performing the identified task; and providing the task transfer report to the second staff member.

F2. The method of F1, wherein the step of identifying the second staff member is based in part on a dynamic attribute of the second staff member.

F3. The method of F2, wherein the dynamic attribute comprises a workload.

G1. A method of handling a task, comprising steps of detecting an initiating event, identifying a first task associated with the detected event, identifying a first staff member to whom the first task should be assigned, identifying a second task that needs to be reassigned from the first staff member to a second staff member, identifying the second staff member based in part of a dynamic attribute of the second staff member, and reassigning the second task to the second staff member.

G2. The method of G1, wherein the second task needs to be reassigned from the first staff member to the second staff member in order for the first staff member to handle the first task within a first response time window associated with the first task.

G3. The method of G2, wherein the second task second task has a second response time window that overlaps the first response time window such that the first staff member cannot complete the second task if the first staff member starts the second task after the first response time window ends.

H1. A method of reducing a fatigue level of a staff member, comprising steps of determining that the fatigue level of a first staff member exceeds a first threshold; identifying an option for reducing the fatigue level of the first staff member, wherein the option comprises reassigning a task from the first staff member; selecting the task to be re-assigned; identifying a second staff member based in part on a first dynamic attribute of the second staff member; and reassigning the task from the first staff member to the second staff member.

H2. The method of H1, wherein the fatigue level is based in part on a second dynamic attribute of the first staff member.

H3. The method of H2, wherein the fatigue level is based in part on a workload.

There are no “I” embodiments.

J1. A method of managing breaks, comprising steps: selecting one or more first clinicians (FCs) to take a concurrent break; identifying a total pool of patients that will need to be covered while the selected FCs are on the concurrent break; selecting one or more second clinicians (SCs) that each have a set of break coverage attributes; assigning one or more of the total pool of patients to each of the selected SCs while complying with their respective break coverage attributes; and determining a remaining pool of patients by removing the patients assigned to the selected SCs from the total pool of patients; and notifying, if the number of patients in the remaining pool of patients is zero, the selected FCs to go on the concurrent break and the selected SCs of the patients that they are respectively assigned to cover during the concurrent break.

J2. The method of J1, wherein the set of break coverage attributes comprises one or more of: a maximum patient-staff ratio; a first maximum patient acuity; a first maximum number of patients having an acuity that is less than or equal to a first threshold acuity; a maximum aggregate patient acuity; a maximum distance between assigned patients; a first maximum number of tasks to be completed during the concurrent break; and a Do Not Disturb (DND) status.

J3. The method of J2, wherein: the set of break coverage attributes further comprises a second maximum number of patients having an acuity that is less than or equal to a second threshold acuity; the second acuity being lower than the first acuity; and the second maximum number of patients is greater than the first maximum number of patients.

J4. The method of J2, wherein: the one or more SCs comprise a Break Nurse (BN); and the step of assigning one or more of the total pool of patients to each of the selected SCs comprises first assigning patients to the BN up to the BN's maximum patient-staff ratio then assigning patients to the other SCs.

J5. The method of J4, further comprising: identifying, if the number of patients in the remaining pool of patients is greater than zero, a Charge Nurse (CN) who is able to cover at least one of the total pool of patients, the CN having a respective set of break coverage attributes; and assigning one or more of the total pool of patients to the CN and then executing the step of assigning one or more of the total pool of patients to each of the selected SCs; wherein: the step of determining a remaining pool of patients comprises removing the patients assigned to the CN from the total pool of patients; and the step of notifying the one or more FCs comprises notifying the CN of the patients that the CN is assigned to cover during the concurrent break.

J6. The method of J5, wherein: the break coverage attributes of the CN comprise a second maximum number of tasks to be completed during the concurrent break if an assistant is assigned to the CN; and the second maximum number of tasks is greater than the first maximum number of tasks.

J7. The method of J5, further comprising: notifying, if the number of patients in the remaining pool of patients is greater than zero, a supervisor that the selected FCs cannot take a concurrent break due to lack of available coverage.

K8. A non-volatile, machine readable memory comprising instructions that, when loaded into a processor and executed, implement steps: selecting one or more first clinicians (FCs) to take a concurrent break; identifying a total pool of patients that will need to be covered while the selected FCs are on the concurrent break; selecting one or more second clinicians (SCs) that each have a set of break coverage attributes; assigning one or more of the total pool of patients to each of the selected SCs while complying with their respective break coverage attributes; determining a remaining pool of patients by removing the patients assigned to the selected SCs from the total pool of patients; and notifying, if the number of patients in the remaining pool of patients is zero, the selected FCs to go on the concurrent break and the selected SCs of the patients that they are respectively assigned to cover during the concurrent break.

L15. A system comprising: a processor; and a non-volatile, machine readable memory coupled to the process, the memory comprising instructions that, when loaded into the processor and executed, implement steps: selecting one or more first clinicians (FCs) to take a concurrent break; identifying a total pool of patients that will need to be covered while the selected FCs are on the concurrent break; selecting one or more second clinicians (SCs) that each have a set of break coverage attributes; assigning one or more of the total pool of patients to each of the selected SCs while complying with their respective break coverage attributes; determining a remaining pool of patients by removing the patients assigned to the selected SCs from the total pool of patients; and notifying, if the number of patients in the remaining pool of patients is zero, the selected FCs to go on the concurrent break and the selected SCs of the patients that they are respectively assigned to cover during the concurrent break.

All structural and functional equivalents to the elements of the various aspects described throughout this disclosure are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.”

This application includes description that is provided to enable a person of ordinary skill in the art to practice the various aspects described herein. While the foregoing has described what are considered to be the best mode and/or other examples, it is understood that various modifications to these aspects will be readily apparent to those skilled in the art, and the generic principles defined herein may be applied to other aspects. Although embodiments of the present disclosure have been described and illustrated in detail, it is to be clearly understood that the same is by way of illustration and example only and is not to be taken by way of limitation, the scope of the present invention being limited only by the terms of the appended claims.

What is claimed is:

1. A system, comprising:
a processor; and
a non-volatile, machine-readable memory coupled to the processor, the memory comprising instructions that, when loaded into the processor and executed by the processor, cause the processor to execute steps of:
retrieving, from a staff management computer system, a work schedule for a set of clinicians currently working in a common care area;
selecting two or more first clinicians (FCs) from the set of clinicians to take a concurrent break;
identifying a total pool of patients currently assigned to the FCs;
retrieving, from a dynamic attribute computer database, break coverage attributes respectively associated with a portion of the set of clinicians;
retrieving, from a task and attribute management computer system, break rules;
selecting one or more second clinicians (SCs) from the portion of the set of clinicians based in part on an evaluation of the break coverage attributes and the break rules; and
assigning one or more of the total pool of patients to each of the selected SCs while complying with their respective break coverage attributes.

2. The method of claim 1, further comprising:
determining a remaining pool of patients by removing the patients assigned to the selected SCs from the total pool of patients; and
notifying, if the number of patients in the remaining pool of patients is zero, the selected FCs to go on the concurrent break and the selected SCs of the patients that they are respectively assigned to cover during the concurrent break.

3. The method of claim 1, wherein the break coverage attributes comprise one or more of:
a maximum patient-staff ratio;
a first maximum patient acuity;
a first maximum number of patients having an acuity that is less than or equal to a first threshold acuity;
a maximum aggregate patient acuity;
a maximum distance between assigned patients;
a first maximum number of tasks to be completed during the concurrent break; and
a Do Not Disturb (DND) status.

4. The method of claim 2, wherein:
the break coverage attributes further comprises a second maximum number of patients having a second acuity that is lower than the first acuity; and
the second maximum number of patients is greater than or equal to the first maximum number of patients.

5. The method of claim 2, wherein:
the one or more SCs comprise a Break Nurse (BN) having respective break coverage attributes; and
the step of assigning one or more of the total pool of patients to each of the selected SCs comprises first assigning patients to the BN up to the BN's maximum patient-staff ratio then assigning patients to the other SCs.

6. The method of claim 4, further comprising:
identifying, if the number of patients in the remaining pool of patients is greater than zero, a Charge Nurse (CN) who is able to cover at least one of the total pool of patients, the CN having respective break coverage attributes; and
assigning one or more of the total pool of patients to the CN and then executing the step of assigning one or more of the total pool of patients to each of the selected SCs;
wherein:
determining the remaining pool of patients comprises removing the patients assigned to the CN from the total pool of patients; and
notifying the one or more FCs comprises notifying the CN of the patients that the CN is assigned to cover during the concurrent break.

7. The method of claim 5, wherein:
the break coverage attributes of the CN comprise a second maximum number of tasks to be completed during the concurrent break if an assistant is assigned to the CN; and
the second maximum number of tasks is greater than the first maximum number of tasks.

8. The method of claim 5, further comprising:
notifying, if the number of patients in the remaining pool of patients is greater than zero, a supervisor that the selected FCs cannot take a concurrent break due to lack of available coverage.

9. The method of claim 1, wherein:
retrieving a work schedule comprises retrieving work schedule information from a staff management system;
retrieving break coverage attributes comprises retrieving break coverage attribute information from a staff management system; and
retrieving break rules comprises retrieving break rule information from a hospital policies system.

10. A non-volatile, machine-readable memory comprising instructions that, when loaded into a processor and executed by the processor, cause the processor to execute steps:
retrieving, from a staff management computer system, a work schedule for a set of clinicians currently working in a common care area;
selecting two or more first clinicians (FCs) from the set of clinicians to take a concurrent break;
identifying a total pool of patients currently assigned to the FCs;
retrieving, from a dynamic attribute computer database, break coverage attributes respectively associated with a portion of the set of clinicians;
retrieving, from a task and attribute management computer system, break rules;
selecting one or more second clinicians (SCs) from the portion of the set of clinicians based in part on an evaluation of the break coverage attributes and the break rules; and
assigning one or more of the total pool of patients to each of the selected SCs while complying with their respective break coverage attributes.

11. A method, comprising steps:
retrieving, from a staff management computer system, a work schedule for a set of clinicians currently working in a common care area;
selecting two or more first clinicians (FCs) from the set of clinicians to take a concurrent break;
identifying a total pool of patients currently assigned to the FCs;
retrieving, from a dynamic attribute computer database, break coverage attributes respectively associated with a portion of the set of clinicians;
retrieving, from a task and attribute management computer system, break rules;
selecting one or more second clinicians (SCs) from the portion of the set of clinicians based in part on an evaluation of the break coverage attributes and the break rules; and
assigning one or more of the total pool of patients to each of the selected SCs while complying with their respective break coverage attributes.
